(19) United States
(12) Patent Application Publication
(10) Pub. No.: US 2020/0199647 A1
(43) Pub. Date: Jun. 25, 2020

(54) ASSAY FOR SCREENING MODULATORS OF HOLOPHOSPHATASE ACTIVITY

(71) Applicant: UNITED KINGDOM RESEARCH AND INNOVATION, Swindon (GB)

(72) Inventors: Anne BERTOLOTTI, Cambridge (GB); Marta CARRARA, Cambridge (GB)

(21) Appl. No.: 16/624,569

(22) PCT Filed: Jun. 21, 2018
(86) PCT No.: PCT/GB2018/051741

§ 371 Date: Dec. 19, 2019

(30) Foreign Application Priority Data
Jun. 21, 2017 (GB) 1709927.6

(51) Int. Cl. C12Q 1/42; C12N 9/16

(52) U.S. Cl. CPC C12Q 1/42; C12N 9/16

(57) ABSTRACT

This invention relates to assays for screening test compounds for their ability to modulate holophosphatase activity.

### FIELD OF THE INVENTION

This invention relates to assays for screening test compounds for their ability to modulate holophosphatase activity.

### BACKGROUND OF THE INVENTION

The reversible phosphorylation of proteins controls the majority of cellular functions. However, while protein kinases have been popular drug targets, phosphatases have generally not been considered viable targets due to their apparent non-specificity.

There are more than 200 holophosphatases in mammals which could, in principle, be drug targets. These holophosphatases are generally made up of a protein catalytic domain with phosphatase activity which binds to one or more regulatory subunits to form/assemble oligomeric specific holophosphatase complexes (or holophosphatases).

Protein phosphatase 1c (PP1c), for example, is a single-domain protein, catalytic subunit that assembles with one, two or more amongst more than 200 diverse regulatory subunits to form specific holophosphatase complexes (Bollen et al., 2010; Choy et al., 2012). In cells, there is no free PP1c, as this would be toxic due to the broad substrate specificity of free PP1c. Instead, within cells regulatory subunits form complexes with PP1c to restrict the specificity of PP1c to cognate substrates, thereby avoiding uncontrolled and promiscuous dephosphorylation events which would be lethal. However, the function of regulatory subunits is not generally well understood.

Attempts to develop inhibitors of holophosphatases have focused on targeting the catalytic subunit. However, inhibition of the catalytic component of a holoenzyme, such as PP1c, results in inhibition of the many (e.g. hundreds) of holophosphatases sharing the same catalytic subunits and may be toxic. Since selectivity is an important property for drug development, the promiscuity of catalytic phosphatases has led them to acquire the reputation of being undruggable.

Regulatory subunits of phosphatases are intrinsically disordered (Bollen et al., 2010; Choy et al., 2012) and are therefore, difficult to express and are generally unstable. Amongst the approximately 200 mammalian PP1 (protein phosphatase 1) holophosphatases, only twelve have been crystallized. There is therefore a lack of structural information on holophosphatases which means that structure-based drug design is not easily applicable to this class of enzyme. To date, the holophosphatases for which structural information is available only contain a small peptide (less than approximately 100 amino acids) from the regulatory subunits (Bollen et al., 2010; Choy et al., 2012) and these smaller structures make it difficult to guide drug discovery. Besides, the crystallized region of the regulatory subunit is the region binding to PP1 which is conserved. Inhibitors targeting this conserved region are unlikely to be selective.

In addition, existing enzymatic assays based on hydrolysis of artificial substrates will mostly lead to the discovery of catalytic inhibitors which are generally not selective.

Accordingly, there is a need to develop a defined holophosphatase activity assay to enable the selection of specific inhibitors or activators of holophosphatases.

It is against this background and the technical problems mentioned herein that the present invention has come about.

### SUMMARY OF THE INVENTION

The present applicants have sought to reconstitute functional holophosphatases using recombinant proteins and have undertaken a series of assays in order to reveal the molecular mechanisms of their selective function and their selective inhibition by small molecule inhibitors. These successful assays have enabled further adaption and testing to develop methods to select or identify compounds that are modulators of holophosphatases. Such methods have far wider application to holophosphatases generally than the specific holophosphatase model exemplified, enabling platform methods for selecting compounds for selective holophosphatase activity modulation.

In a first aspect the invention provides a method of screening a test compound for modulation of holophosphatase activity comprising:
a) providing a functional and selective holophosphatase comprising a catalytic subunit and at least one regulatory subunit;
b) providing a phosphorylated protein substrate;
c) combining the phosphorylated substrate and the holophosphatase and incubating together in the presence or absence of a test compound;
d) measuring dephosphorylation;
   wherein a variation in dephosphorylation in the presence of the test compound as compared to in the absence of the test compound indicates that the test compound is a modulator of holophosphatase activity.

By “modulation of holophosphatase activity” is meant the ability of a test compound to modulate holophosphatase activity. In one embodiment, the “modulation of holophosphatase activity” is inhibition of holophosphatase activity. In another embodiment, the “modulation of holophosphatase activity” is the activation of holophosphatase activity.

Suitably the holophosphatase activity that is modulated by the test compound is selective holophosphatase activity.

In one embodiment a modulator of holophosphatase activity inhibits the dephosphorylation activity and is therefore an inhibitor of holophosphatase activity. For an inhibitor, a decrease in dephosphorylation in the presence of the test compound as compared to in the absence of the test compound indicates that the test compound is an inhibitor of holophosphatase activity. In another embodiment, a modulator of holophosphatase activity increases the dephosphorylation activity of the holophosphatase and is therefore an activator of holophosphatase activity. For an activator an increase in dephosphorylation in the presence of the test compound as compared to in the absence of the test compound indicates that the test compound is an activator of holophosphatase activity. FIG. 17C demonstrates how this may be determined.

Advantageously, the method in accordance with the present invention provides a measure of selective inhibition of holophosphatases. Previous methods could only reveal inhibition of the catalytic phosphatase/catalytic subunit by small molecules. More specifically, before this invention, there was no method to measure the selective inhibition of holophosphatases with selective modifiers targeting their regulatory subunits

By “functional” is meant a holophosphatase capable of showing dephosphorylation activity at a concentration range below the range for which the isolated catalytic domain alone shows dephosphorylation. By “selective” is meant a holophosphatase capable of selective dephosphorylation activity when incubated with a cognate phosphorylated protein substrate but incapable of dephosphorylating a non-cognate substrate. Thus the selectivity of the holophosphatase is determined by its selectivity for a substrate, suitably a cognate substrate (i.e. the known preferred substrate of a particular holophosphatase), or a group of such substrates, and its ability to dephosphorylate the given substrate or group of substrates but not any substrates, in particular not non-cognate substrates (i.e. those phosphorylated molecules which are not known to interact with a particular holophosphatase). This differentiates selective holophosphatases from their isolated catalytic subunits which can dephosphorylate nearly any substrate without selectivity.

In one embodiment, the holophosphatase for use in the method of screening in accordance with the invention is prepared by purifying a holophosphatase. Suitable sources for purifying a holophosphatase include from cell culture or tissue samples. Tissues can be from any source including animal or plant tissue sources. Suitable animal sources include bovine (e.g. kidney, brain), rabbit etc. Other suitable sources will be familiar to the skilled person. In one embodiment, the subunits may be endogenous proteins purified from a cell extract or a tissue by any suitable method, for example by chromatography.

In another embodiment, of any of the protein components for the method of the present invention (i.e. catalytic and regulatory subunits, phosphorylated substrate) are recombinant proteins. Suitably, the holophosphatase may be a recombinant protein which may be synthesised by expressing the catalytic subunit and the at least one regulatory subunit in any system, such as a prokaryotic or eukaryotic cell system, so as to generate a functional reconstituted form. In one embodiment, the catalytic and regulatory subunits may be expressed in separate cell systems, and optionally purified, before combining in vitro under suitable conditions for reconstitution of the functional holophosphatase. Alternatively, the different subunits can be co-expressed in a suitable expression system (e.g. bacterial, insect or mammalian cell) and the holophosphatase can be purified by any suitable method (for example chromatography).

In one embodiment, the recombinant proteins for use in the method are not naturally expressed by a micro-organism. In other embodiments, recombinant proteins are expressed from foreign nucleic acid by introducing DNA into a biological sample or animal. In other embodiments, the recombinant proteins are expressed by bacterial, mammalian or insect cells. In another embodiment, proteins are expressed in heterologous cell-free systems such as reticulocyte lysates or wheat germ lysates.

Regulatory subunits are natively unstructured making them generally difficult to express in a functional form. Advantageously, using shorter fragments can overcome problems in low protein yields and low stability observed using the full length proteins. Accordingly, in one embodiment, the reconstituted form may comprise a regulatory subunit which is a truncated fragment of the naturally occurring regulatory subunit. Suitably said truncated fragment further comprises a region of the regulatory subunit which binds to the catalytic subunit. The truncated fragment may also comprise a region of the regulatory subunit which binds to or permits recognition of the phosphorylated protein substrate. Suitably, said truncated version or fragment of the full length subunit comprises a region of the regulatory subunit for binding to a modulator i.e. an inhibitor or activator. In one embodiment, the region may be a region known to bind to known modulators e.g. inhibitors. Such known inhibitors may include compounds such as Guanabenz, Sephin 1, and Raphin 1 (TST3) (described, for example, in WO2014108520A1, WO2016162688A1 and WO2016162689A1), for example. In other embodiments, the region which binds to a modulator e.g. an inhibitor may be different to the region which binds to known modulators e.g. inhibitors.

Thus, such truncated versions of fragments of the regulatory subunit retain the ability to bind the catalytic subunit, an inhibitor and the protein substrate. In one embodiment, when no inhibitors/activators are known, the suitable fragment of regulatory subunit needed for the functional assay comprises the region which binds to the catalytic subunit and also the region which binds to the substrate. In other embodiments, the largest possible functional fragment of regulatory subunit may be used, or even more preferably, the full length protein. As such, the version of regulatory subunits are functional and inhibitable and can be used to search for inhibitors or activators using functional and biochemically defined assays, as described herein.

It will be understood that a functional and/or selective holophosphatase for use in the method of the present invention may comprise a recombinant or mutant protein catalytic subunit or regulatory subunit, comprising one or more amino acid substitutions, insertions or deletions compared to the wild-type form of the holophosphatase, providing the catalytic subunit and the at least one regulatory subunit may be used to generate a functional reconstituted form. Suitably a catalytic subunit may be a Ser/Thr phosphoprotein phosphatase (PPP) including any one of the PPP superfamily such as any one of the Ser/Thr protein phosphatases 1-7 (PP1-7) (described, for example, by Heroes et al., 2012). Thus, but without limiting to the following examples, suitable catalytic subunits include PP1, PP2, PP3, PP4, PP5, PP6 and PP7 holophosphatases. For example, suitable human holophosphatase catalytic subunits may include PPP1CA, PPP1CIB, PPP1CC, PPP2CA, PPP2CB, PPP3CA, PPP3CB, PPP3CC, PPP4C, PPP5C or PPP6C. In one embodiment, the catalytic subunit is PP1c.

Suitable regulatory subunits include Ser/Thr phosphoprotein phosphatase interacting proteins. In particular the regulatory subunit is a protein which interacts with the corresponding PPP. For example, where the catalytic subunit is a PP1 family phosphoprotein phosphatase, the regulatory subunit will be a PP1-interacting protein (PIP). Over 200 PIPs have been identified in vertebrates to date. Examples of suitable PIPs are given, for example, in ((Heroes et al., 2012); see pages 585-586, Table 1). In one embodiment, the regulatory subunit is selected from R15A and R15B, or fragments thereof. Amino acid sequences for R15A and R15B are given in WO2016162688A1. Suitable fragments include R15A³²⁵⁻⁶³⁶ and R15B³⁴⁰⁻⁶⁹⁸ or those fragments comprising +/−approximately 10 amino acids thereof, and comprising the functional regions as set out above i.e. catalytic subunit-binding, inhibitor-binding and substrate-binding regions.

Fragments comprising +/−approximately 10 amino acids thereof encompasses fragments approximately 10 amino acids longer than R15A³²⁵⁻⁶³⁶ or R15B³⁴⁰⁻⁶⁹⁸ at the amino-terminus, at the carboxy-terminus, or at both the amino- and carboxy-termini, and fragments approximately 10 amino acids or less shorter than R15A³²⁵⁻⁶³⁶ or R15B³⁴⁰⁻⁶⁹⁸ at the amino-terminus, at the carboxy-terminus, or at both the amino- and carboxy-termini. In another embodiment, a suitable fragment of R15A may comprise approximately amino acids 463-636. In one embodiment, the region of the regulatory subunits included in the assay is sufficient for binding inhibitors or activators. In one embodiment, where the PIP is selected from R15A and R15B, these are located at the amino-terminal region which permits recognition of a specific phosphatase protein substrate. Thus in other embodiments, the fragments of enzyme regulatory subunits may comprise regions that are selected from R15A^{N} (R15A³²⁵⁻⁵¹²), R15A^{C} (R15A⁵¹³⁻⁶³⁶), R15B^{N} (R15B³⁴⁰⁻⁶³⁵) and R15B^{C} (R15B⁶³⁶⁻⁶⁹⁸).

In one embodiment, the region is sufficient for allosteric regulation. In one embodiment, an allosteric inhibitor may be identified which alters substrate recruitment, as described herein in the examples section for the known inhibitors. However, it will be appreciated that other allosteric inhibitors may perturb other aspects of the holoenzyme function.

Suitable phosphorylated protein substrates will be those substrates which are known to be substrates for the particular holophosphatase of interest. Thus, suitably the phosphorylated protein substrate will be chosen to be a cognate/preferred substrate for a particular holophosphatase.

Such protein substrates can include a specific phospho-protein or a fragment thereof such as a phospho-peptide which mimics a sufficient proportion of the natural substrate of the holophosphatase. As described herein, eIF2alpha is a suitable substrate for R15A-PP1c and/or R15B-PP1c. In one embodiment the phosphorylated protein substrate is purified. In one embodiment, the phosphorylated protein substrate is labelled with a detectable label such as, for example, a radioactive label or any other method suitable to detect phosphorylation. In other embodiments, the phosphorylation status of the protein substrate may be detected using antibodies which recognise the phosphorylated form for detection in an immunoassay such as an immunoblot, or ELISA or similar (AlphaLisa) or Luminex or FRET. Examples of suitable detection methods are described herein.

In one embodiment, the method of screening comprises providing a holophosphatase comprising a catalytic subunit wherein the catalytic subunit is provided at a concentration which is sub stoichiometric to the concentration of the substrate. Suitably, the catalytic subunit is provided at physiological concentration. In another embodiment, the catalytic subunit is provided at a low concentration; suitably said low concentration is determined experimentally for each particular regulatory subunit as being that concentration at which the catalytic subunit is inactive in dephosphorylating a given substrate in the absence of that particular cognate regulatory subunit. In other words, a “low concentration” is where, in the presence of the regulatory subunit, the holophosphatase shows selective dephosphorylation activity. Such selective activity can be determined in assays using suitable controls e.g. cognate/non-cognate substrates, irrelevant subunits, catalytic subunit alone. Suitable methods and controls are exemplified herein and with particular reference to FIG. 17. In one embodiment, where the catalytic subunit is PP1c, it is provided at a concentration of less than 1 micromolar (1 μM), preferably 0.2 μM, preferably less than 100 nM, preferably 50, 40, 30, 20 or 10 nM. In the examples as described herein for example 1 micromolar (1 μM) PP1c dephosphorylates substrates non-selectively. At 10 nM, PP1c alone does not dephosphorylate the substrate eIF2α. At 10 nM, the R15-PP1c holophosphatases dephosphorylate their cognate substrate eIF2α but not a non-cognate substrate, phosphorylase a. Also, at 10 nM, an unrelated holophosphatase PP1c-R3A does not dephosphorylate eIF2α.

Suitably the method of screening is in vitro; cellular assay; biochemical assay/cell free assay. In one embodiment, one or more of the protein components of the screening method may be expressed with an affinity tag (for pull-down). Suitable tags will be familiar to those skilled in the art and include, for example, affinity tags such as Maltose Binding Protein-tag, Glutathione S-transferase, Histidine tags etc.

In one embodiment, the method for screening a test compound further comprises performing a confirmatory assay to test for a particular modulatory i.e. inhibitory/activatory activity. For example, a test inhibitor may be validated in an enzymatic assay or a cell based assay to demonstrate target engagement. Suitable assays for activity of particular holophosphatases will depend on the particular cell pathways in which they are involved. The skilled person will be aware of suitable assays for any particular holophosphatase in the knowledge of the molecules with which the holophosphatase interacts as substrates. Suitable cell-based assays to measure activity of holophosphatase inhibitors and target engagement in cells are disclosed in WO2016162688A1.

Suitably a test compound binds to the regulatory subunit. In one embodiment, the test compound binds to the regulatory subunit and induces a conformational change in the regulatory subunit. In one embodiment, the test compound is an allosteric inhibitor.

While others have proposed that the R15 inhibitors Sephin1 and Guanabenz should disrupt the protein-protein interaction between PP1 and R15 (Choy et al., 2015; Crespillo-Casado et al., 2017), it was previously not anticipated that R15 inhibitors induce a selective conformational change in their target. The present Examples also demonstrate that Guanabenz and Sephin1 are selective inhibitors of R15A, and Raphin1 is a selective inhibitor of R15B. Guanabenz, Sephin1 and Raphin1 are selective inhibitors of their respective target: in this instance they induce a conformational change in R15. Thus it is anticipated that this represents a generic mechanism and more modifiers of regulatory subunits will be found that inhibit or activate their function by changing their conformation.

Accordingly in another aspect of the invention there is provided a method for screening for an inhibitor of a holophosphatase comprising providing a test compound and a holophosphatase regulatory subunit under conditions for binding the compound and the regulatory subunit, and detecting a conformational change in the regulatory subunit upon binding of an inhibitor. The conformational change can be detected by any method suitable to detect conformational change of proteins. The skilled person will be aware of suitable assays. For example, a conformational change is detected by limited proteolysis observed upon incubation of a protein and limited concentration of a protease in the presence of a test compound, as shown in the example here. If a test compound induces a conformational change, it alters the sensitivity of the protein to mild protease degradation. The compound can either render the protein more sensitive to proteolysis or more resistant. The skilled person will know how to empirically determine the concentration of the protease needed for the assay as well as the time of incubation such that the proteolysis is not complete but limited. In one embodiment, the protease may be trypsin or any other protease such as chymotrypsin, V8, thrombin, thermolysin, pepsin, Lys-C, Lys-N, caspase 1, caspase 2, caspase 3, caspase 4, caspase 5, caspase 6, caspase 7, caspase 8, caspase 9, caspase 10, Arg-C, Asp-N, clostripain, Factor Xa, granzyme B, Proteinase K, CNBR, hydroxylamine, enterokinase, glutamyl endopeptidase, tobacco etch-virus protease, proline endopeptidase.

In other embodiments a conformational change may be detected by any other suitable evaluation methods, FRET, BRET, NMR, crystallisation, or any method known to detect conformational changes of proteins.

In another aspect there is provided a method of synthesising a reconstituted functional and selective holophosphatase as described herein.

Suitably, said regulatory subunit, or fragment thereof, comprise(s) sufficient regions of the regulatory subunit for both interaction with the catalytic subunit and recognition of a specific phosphorylated protein substrate.

In one aspect, there is provided a reconstituted functional holophosphatase produced in accordance with a method of the invention.

In another aspect there is provided a reconstituted functional holophosphatase selected from PP1, PP2A, PP2B, PP3, PP4, PP5 and PP6 or any other suitable phosphatase having a regulatory subunit comprising binding to the catalytic subunit, binding domains sufficient for recognition of the protein substrate, in combination with an inhibitor or activator binding domain.

In another aspect there is provided a reconstituted functional PP1 holophosphatase having a regulatory subunit comprising binding domains at the carboxy-terminal region sufficient for interaction with the catalytic subunit and binding domains at the amino-terminal region sufficient for recognition of its eIF2α protein substrate. Suitably, in this aspect, the regulatory subunit is selected from R15A or R15B. In one embodiment, in the reconstituted functional PP1 holophosphatase in accordance with this aspect of the invention the regulatory subunit comprises at least R15A³²⁵⁻⁶³⁶, or the regulatory subunit comprises at least R15B³⁴⁰⁻⁶⁹⁸.

In another aspect there is provided a kit for screening test compounds for selective holophosphatase inhibition activity comprising:

a functional and selective holophosphatase in accordance with the invention, or made in accordance with a method in accordance with the invention; and

a purified labelled phosphorylated holophosphatase protein substrate.

Suitably the kit in accordance with the invention is for identifying a test compound as a drug candidate.

In other aspects, the invention provides a method of screening for holophosphatase modulators based on the competition of known inhibitors of holophosphatases in accordance with the invention. Such a compound may be selected for its virtue to interact with the binding domain of the regulatory subunit. Suitably, the test compound may compete with the known inhibitor(s) of particular regulatory subunits in an competition assay. Suitably said compound can be an allosteric inhibitor of a regulatory subunit.

In other aspects, the invention provides a test compound which is an inhibitor of a holophosphatase obtained by a method of screening in accordance with the invention. Such a compound may be designed to interact with the a known inhibitor binding domain of the regulatory subunit. Suitably, the test compound may compete with the known inhibitor(s) of particular regulatory subunits in a competition assay.

### BRIEF DESCRIPTION OF THE FIGURES

Certain embodiments of the present invention will now be described, by way of example only, with reference to the drawings in which:

FIG. 1 Reconstitution of functional eIF2α holophosphatases with recombinant proteins. (A) Immunoblots showing P-eIF2α and eIF2α following a dephosphorylation reaction, of 1 μM P-eIF2α, by recombinant PP1 (1 μM) in the presence or absence of 1 μM of recombinant R15A, R15B or R3A. (B-D) Immunoblots of P-eIF2α and eIF2α following a dephosphorylation reaction, of 1 μM P-eIF2α, by PP1 (1 μM) in the presence or absence of (B) R15A and its inhibitors Guanabenz or Sephin1, (C) R15B and its inhibitor Raphin1 and (D) R15A or R15B with Calyculin A. (E) Titration curve of P-eIF2α (1 μM) dephosphorylation by increasing PP1 concentrations. A representative immunoblot corresponding to this titration is shown in FIG. 7B. Data are means±SEM (n=3). (F-H) Immunoblots of P-eIF2α and eIF2α following a dephosphorylation reaction, of 1 μM P-eIF2α, by PP1 (10 nM) in the presence or absence of 1 μM (F) R15A, (G) R15B, or (H) R3A. All dephosphorylation reactions were carried out at 30° C. for 16 h. For all experiments, representative results of three independent experiments are shown (n=3; biological replicates).

FIG. 2 Defining the R15 domains required for PP1c binding and eIF2α holophosphatase activity. (A, B) Schematics of the proteins (A) R15A and (B) R15B. Amino acid residues delimiting the amino-terminal, carboxy-terminal regions and location of MBP and His₆ affinity tags are shown. The location of the PP1c binding region is indicated. (C, D) Thermophoresis binding curves of labelled PP1c binding to titrations of unlabelled (C) R15A (amino acids 325-636), R15A^{N} (amino acids 325-512; the amino-terminal fragment), R15A^{C} (amino acids 513-636; the non-functional carboxy-terminal fragment), (D) R15B (amino acids 340-698), R15B^{N} (amino acids 340-635; the amino-terminal fragment), R15B^{C} (amino acids 636-698; the non-functional carboxy-terminal fragment). Dissociation constants (KD) are means±SEM (n=3). (E, F) Immunoblots of P-eIF2α and eIF2α following a dephosphorylation reaction by PP1c (10 nM) in the presence or absence of (E) R15A, R15A^{N}, R15A^{C}, (F) R15B, R15B^{N}, R15B^{C}. Dephosphorylation reactions were carried out at 30° C. for 16 h.

FIG. 3 R15 holophosphatases have a higher affinity for P-eIF2α than PP1c. (A) Thermophoresis binding curve of labelled P-eIF2α binding to titrations of unlabelled PP1c^{D95A}. Dissociation constant (K_{D}) is the mean±SEM (n=3). (B-D) Thermophoresis binding curves of labelled P-eIF2α binding to titrations of unlabelled (B) R15A, R15A^{N}, R15A^{C}, (C) R15B, R15B^{N}, R15B^{C}. (D) R3A measured by thermophoresis. Dissociation constants (KD) are means±SEM (n=3; biological replicates). (E) Dissociation constants (K_{D}) of labelled P-eIF2α to titrations of unlabelled PP1c^{D95}A, in the presence of saturating, and unlabelled, functional R15 (R15A or R15B), their non-functional carboxy-terminal fragments (R15A^{C} or R15B^{C}), or the amino-terminal fragments of R15s (R15A^{N} or R15B^{N}). The amino-terminal fragments of R15s do not bind to PP1c (FIG. 2C, D) and are included as negative controls. K_{D} values shown are means±SEM (n=3) and values are indicated in Table 1. R3A is an irrelevant regulatory subunit which inhibited dephosphorylation of eIF2α by PP1c (FIG. 1A). The value of P-eIF2α binding to PP1c^{D95A} corresponds to (A).

FIG. 4 R15 inhibitors decrease eIF2α binding to their selective R15 target. (A) Binding of R15A to biotinylated-Guanabenz and biotinylated-Sephin1 immobilized on Streptadvidin beads. Immunoblots of input and bound samples, probed with α-MBP (to reveal R15s) are shown. Bound samples (lane 3) were eluted with an excess of Guanabenz or Sephin1, respectively. Representative results of three independent experiments are shown (n=3; biological replicates). (B,C) Coomassie-stained gels showing limited trypsin digestion of (B) R15A and (C) R15B in the presence or absence of Guanabenz, Sephin1 or C3. Trypsin digestions were carried out using 2.5 nM of trypsin, and reactions were allowed to proceed for 0 h (first lane in each gel), 30 min, 1 h, 2 h and 3 h at 22° C. Reactions were terminated by addition of 4% SDS Leammli sample buffer. Representative results of three independent experiments are shown (n=3; biological replicates). (D) Coomassie-stained gels showing limited trypsin digestion of R15s in the presence or absence of Raphin1. Trypsin digestions were carried out using 5 nM of trypsin, and reactions were allowed to proceed for 5 min at 22° C. Reactions were terminated by the addition of 4% SDS Laemmli sample buffer. (E-G) Binding of P-eIF2α to MBP-tagged R15s immobilized on magnetic amylose beads (see methods) in the presence or absence of (E) Guanabenz, (F) Sephin1, or (G) Raphin1. Immunoblots of input and bound samples, probed with anti-MBP (to reveal R15s) or anti-eIF2α antibodies are shown.

FIG. 5 An activity assay with functional recombinant R15 holophosphatases recapitulates selective inhibition by R15 inhibitors. (A-C) Immunoblots of P-eIF2α and eIF2α following a dephosphorylation reaction by PP1c (10 nM)-R15A and -R15B holophosphatases in the presence or absence of (A) Guanabenz, (B) Sephin1 or (C) Raphin1. (D, E) Immunoblots of P-eIF2α and eIF2α following a dephosphorylation reaction by 10 nM PP1c-(10 nM) -R15A^{N}/R15B^{C} and -R15B^{N}/R15A^{C} chimeric holophosphatases in the presence or absence of (D) Guanabenz, Sephin1 or (E) Raphin1. All dephosphorylation reactions were carried out at 30° C. for 16 h. (F) Immunoblots of P-eIF2α and eIF2α following a dephosphorylation reaction by PP1c (10 nM) -R15A or R15B holophosphatases in the presence or absence of C3.

FIG. 6 A Coomassie-stained gel showing expressed proteins subsequently purified for use in the methods of the invention. Those shown are eIF2α (lane 1), a large fragment, known to bind Guanabenz and Sephin1 (Das et al., 2015; Tsaytler et al., 2011), of the regulatory subunit R15A³²⁵⁻⁶³⁶ (lane 3), the homologous fragment of R15B (R15B³⁴⁰⁻⁶⁹⁸, lane 4), as well as an unrelated regulatory subunit R3A (PPP1R3A, glycogen-targeting subunit of protein phosphatase 1 (GM), lane 5). PP1 (lane 2) was expressed and purified as previously described (Peti et al., 2013).

FIG. 7. Reconstitution of functional eIF2α holophosphatases with recombinant proteins. (A,D-F) Phos-tag gels of experiments shown in FIG. 1. FIG. 7A,D-F correspond to samples analyzed by immunoblotting shown in FIG. 1A,F-H respectively. Samples were run on 15% Phos-tag gels and visualized by Coomassie staining. (B) Representative immunoblot of P-eIF2α and eIF2α following a dephosphorylation reaction, of 1 μM P-eIF2α, by increasing amounts of PP1 used for the titration curve in FIG. 1E. The concentration of PP1 used is indicated. (C) Phos-tag gel of titration of increasing amounts of PP1 with P-eIF2α substrate. Samples were run on 15% Phos-tag gels and visualized by Coomassie staining. Dephosphorylation reactions were carried out at 30° C. for 16 h. Representative results of three independent experiments are shown (n=3; biological replicates).

FIG. 8. Phosphorimaging and Coomassie gel of ³³P Phosphorylase a following a dephosphorylation reaction, of 1 μM ³³P Phosphorylase a, by PP1 (1 μM and 10 nM) in the presence or absence of 1 μM R15A or R15B. All dephosphorylation reactions were carried out at 30° C. for 16 h. Representative results of three independent experiments are shown (n=3; biological replicates).

FIG. 9. Immunoblot of P-eIF2α and eIF2α following a dephosphorylation reaction by PP1c (10 nM) or PP1c^{D95A} (10 nM) in the presence of R15A, or R15B. All dephosphorylation reactions were carried out at 30° C. for 16 h.

FIG. 10. Dose dependent cytoprotection of HeLa cells by Guanabenz, but not the inactive derivative C3, in response to Tunicamycin stress. Vehicle concentrations represent the corresponding amounts of DMSO used in compound treated samples (from 0 to 0.04% highest concentration). Cell viability after 72 h of treatment was monitored using the IncuCyte ZOOM system. Representative results of three independent experiments are shown (n=3; biological replicates).

FIG. 11. Coomassie-stained gels showing limited trypsin digestion of MBP in the presence or absence of Guanabenz or Sephin1. Trypsin digestions were carried out using 2.5 nM of trypsin. Reactions were allowed to proceed for 0 h (first lane in each gel), 30 min, 1 h, 2 h or 3 h at 22° C. Reactions were terminated by addition of 4% SDS Laemmli sample buffer. Representative results of three independent experiments are shown (n=3; biological replicates).

FIG. 12. Light scattering measurements of 5 μM R15A in the presence of 1 mM Guanabenz, 1 mM Sephin1, 1 mM C3, 50 μM Salubrinal or DMSO vehicle. Absorbance at 380 nm was monitored over 10 min at 20° C. with constant stirring. 100 data points are plotted for each sample.

FIG. 13. Binding of PP1c to MBP-tagged R15s immobilized on magnetic amylose beads (see methods) in the presence or absence of Guanabenz, Sephin1, or Raphin1. Immunoblots of input (A) and bound (B) samples, probed with anti-MBP or anti-PP1cα antibodies are shown.

FIG. 14. (A-E) Immunoblots of P-eIF2α and eIF2α following a dephosphorylation reactions by increasing amounts of PP1c, in the presence of (A) DMSO control, (B) Guanabenz, (C) Sephin1, (D) Raphin1, or (E) Salubrinal. The concentration of PP1c used is indicated. All dephosphorylation reactions were carried out at 30° C. for 16 h.

FIG. 15. (A, B) Images of the effect of adding increasing amounts (from 30 μM up to 2 mM) Salubrinal to (A) P-eIF2α and (B) PP1c. (C, D) Images of the effect of adding 2 mM Guanabenz, Sephin1 or Raphin1 to (C) P-eIF2α and (D) PP1c.

FIG. 16. Phos-tag gel showing P-eIF2α and eIF2α following a dephosphorylation reaction, using free PP1c (10 nM) or PP1c (10 nM) plus R15A. Dephosphorylation reactions were carried out at 30° C. for the time indicated.

FIG. 17. Cartoon showing the methodological paradigm of the invention, representing the set of rules used to define the specific conditions for a functional and selective holophosphatase assay.

### DETAILED DESCRIPTION

The notion that holophosphatases can be selectively inhibited by targeting their regulatory subunits is slowly emerging. Guanabenz was discovered through a phenotypic assay: it protects cells from protein misfolding stress in the endoplasmic reticulum (ER) (Tsaytler et al., 2011). It does so by prolonging the benefit of eIF2α phosphorylation by selectively binding and inhibiting R15A but not the related protein R15B. Sephin1 (disclosed in CA2896976 A1) selectively inhibits R15A but not R15B, whilst devoid of both the α-2 adrenergic activity of Guanabenz (Das et al., 2015; Tsaytler et al., 2011). Sephin1 has suitable properties for in vivo studies and therefore was used to inhibit R15A in mice. Sephin1 is orally available, crosses the blood-brain barrier, and reaches concentrations in the brain known to inhibit R15A (Das et al., 2015). When given to mice, it safely prevents the motor, morphological and molecular defects associated with two otherwise unrelated protein-misfolding diseases: Charcot-Marie-Tooth 1B (CMT-1B) and a SOD1 form of amyotrophic lateral sclerosis (ALS) (Das et al., 2015).

So far, the molecular basis for this selective inhibition of R15A of Guanabenz and Sephin1 has not yet been elucidated. Two groups have tried to reconstitute the selective inhibition of R15A-PP1 in vitro with recombinant proteins but this has not been possible (Choy et al., 2015; Crespillo-Casado et al., 2017). This is because there was no understanding of the function of regulatory subunit nor understanding that selectivity of a holophosphatase is only possible under physiological conditions i.e. when the catalytic subunit is used at sub-stoichiometric concentrations relative to the substrates. Thus, in these earlier attempts, the authors failed to generate a selective holophosphatase and therefore saw no inhibition.

The present applicants previously disclosed in application WO2016/162688 that selective inhibitors of either the inducible R15A (PPP1R15A/GADD34) or the constitutive R15B (PPP1R15B/CReP) regulatory subunit of the eIF2α holophosphatase may be important; these selective inhibitors are potent and orally-available treatments that prevent diverse neurodegenerative diseases in mice. However, the molecular basis for their selective inhibition activity was not elucidated and there are no biochemically defined assays to functionally characterize and select holophosphatase inhibitors. This therefore continues to present a challenge in the advancement of methods which may usefully screen for selective holophosphatase inhibitor drug candidates. Until now no biochemically defined platform method has been available to enable further progress in functional identification of such holophosphatase inhibitors.

Advantageously the method of screening in accordance with the invention provides a method which measures dephosphorylation activity and modulation (i.e. inhibition or activation thereof). The previously described binding assays such as binding to an SPR chip (as described, for example, in WO2016/162688) are useful for identification of selective binders to holophosphatases, however, the modulatory activity of the compounds on the holophosphatase activity cannot be determined by the binding assay alone but only in conjunction with additional assays such as cell assays. Advantageously, the methods and assays of the present invention provide, for the first time, a biochemically defined and selective holophosphatase assay to identify and select modulators of the holophosphatase activity (inhibitors or activators). This is particularly important in the case of the holophosphatase inhibitors which may be identified by the methods as described herein as these can identify selective inhibitors targeting regulatory subunits. It is generally the case that there is a poor correlation between binding affinities and resulting activation or inhibition for allosteric ligands. Thus, the selective activity assay described here is valuable because it will enable drug discovery of new holophosphatase inhibitors targeting the regulatory subunits in one activity assay.

### Determining Conditions for a Functional and Selective Holophosphatase Assay

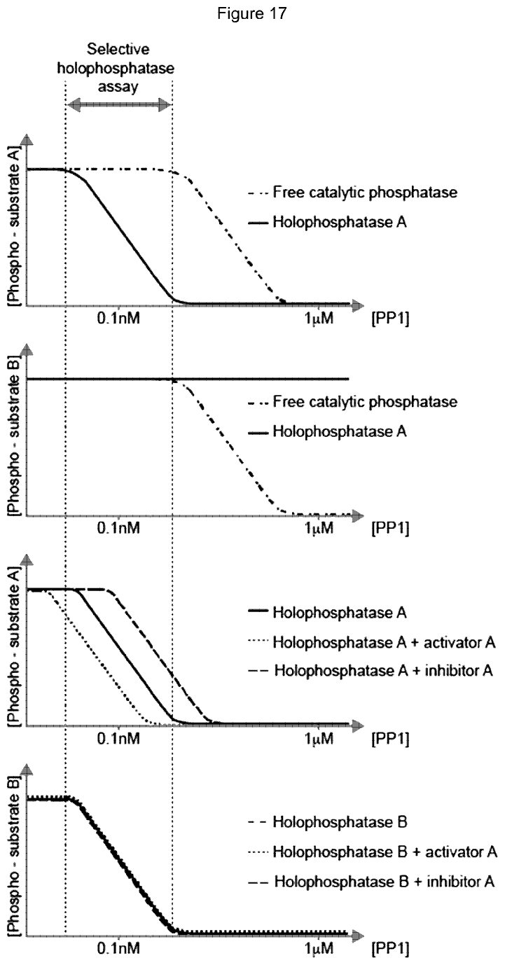

FIG. 17 shows a set of graphs which exemplify how the specific conditions for a particular holophosphatase to be functional and selective can be determined. This allows a set of rules to define the specific conditions for a functional and selective holophosphatase assay to be determined. The activity of the free catalytic subunit (“Free catalytic phosphatase”) of a given holophosphatase (Holophosphatase A) is titrated by measuring dephosphorylation of the cognate phospho-substrate of holophosphatase A, phospho-substrate A, using different concentrations of free catalytic phosphatase (catalytic subunit). Here the y-axis shows the concentration of phospho-substrate ([phospho-substrate]), wherein a decrease in phospho-substrate concentration is indicative of dephosphorylation.

Likewise, the activity of a functional holophosphatase A (“Holophosphatase A”) (composed of a catalytic subunit and a regulatory subunit capable of binding to the catalytic subunit and to the substrate) is titrated by measuring dephosphorylation of its cognate phospho-substrate called phospho-substrate A using different concentrations of Holophosphatase A.

The conditions under which Holophosphatase A is functional and selective are those conditions where dephosphorylation of the phospho-substrate A is achieved by the Holophosphatase A but not by the Free catalytic phosphatase (catalytic subunit). This is indicated in FIG. 17 as the range “Selective holophosphatase assay” shown.

The concentration ranges are indicated by [PP1] on the x-axis. In this Figure, the concentration ranges shown relate to the specific examples of components used for this exemplification i.e. “Free catalytic phosphatase”=PP1c; “Holophosphatase A”=R15A-PP1; “Phospho-substrate A” is eIF2α (i.e. the particular cognate substrate); “Phospho-substrate B” (i.e. the particular control or non-cognate substrate)=phosphorylase a. It will be understood that this example can be extrapolated to any other holophosphatase by substituting the components of the assay. A variation in the components will lead to a variation in the particular concentration range of the holophosphatase (the range indicated as “Selective holophosphatase assay”) for which the holophosphatase activity is considered to be functional and selective.

The activity of the functional holophosphatase A is also titrated by measuring dephosphorylation of one or more irrelevant phospho-substrate (“Phospho-substrate B”) using different concentrations of Holophosphatase A. Thus, the conditions under which holophosphatase is selective may also be those concentrations where Holophosphatase A dephosphorylates the cognate phospho-substrate A but not an irrelevant substrate, phospho-substrate B.

In the example provided in the invention, R15A-PP1 dephosphorylated eIF2α but not an irrelevant substrate phosphorylase a.

Thus, a suitable concentration of holophosphatase for a selective holophosphatase dephosphorylation assay in accordance with the invention is a concentration where the holoenzyme/holophosphatase is active against its cognate substrate (phospho-substrate A) and selective (i.e. inactive against irrelevant substrate B or other) whilst the isolated catalytic subunit alone is inactive. Suitably, a 2 fold difference between the concentration of holophosphatase relative to free catalytic subunit required to dephosphorylate the substrate is desirable, 5 fold difference is preferred, 10 fold or above are even more preferred.

Such a defined and selective assay enables the selection of selective holophosphatase inhibitors or activators. An activator of holophosphatase A will increase the activity of holophosphatase A towards the cognate substrate A. An inhibitor of holophosphatase A will decrease the activity of holophosphatase A towards the cognate substrate A. This is demonstrated in FIG. 17C where the effect of an activator and an inhibitor on the phosphorylation titration curves in the presence of a cognate substrate are demonstrated.

The inhibitors isolated in this assay can then be counter-screened using isolated catalytic subunits or irrelevant holophosphatases. A selective inhibitor of a holophosphatase A does not inhibit holophosphatase B (or the isolated catalytic subunit thereof). Suitably a selective inhibitor of a holophosphatase identified in this assay shows at least a 2 fold selectivity for one holophosphatase over the other, preferable 3 or 5 fold and even more preferable 10 fold or more.

### Test Compounds

A test compound for use in an assay in accordance with any aspect or embodiment of the invention may be a protein or polypeptide, polynucleotide, antibody, peptide or small molecule compound. In one embodiment, the assay may encompass screening a library of test compounds e.g. a library of proteins, polypeptides, polynucleotides, antibodies, peptides or small molecule compounds. Suitable high throughput screening methods will be known to those skilled in the art.

### Kits and Apparatus

In other aspects or embodiments of the invention, kits and/or apparatus arranged for use and/or when used for a screening method in accordance with the invention are provided. Suitable kits and/or apparatus may include surface attachment of a substrate, for example to a chip or solid surface, e.g. a bead or microtitre plate. Suitably a chip or bead may be arranged in such a way as to enable the screening method as described herein to be carried out.

Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above and table described below.

### Examples

A well-established property of PP1c regulatory subunits is to restrict the otherwise broad substrate selectivity of PP1c (Heroes et al., 2012). This has defined regulatory subunits as inhibitors of PP1c because they block the ability of free PP1c to dephosphorylate non-cognate substrates (Hendrickx et al., 2009). Recombinant R15A has been previously characterized using this paradigm and indeed inhibits PP1c from dephosphorylating an irrelevant substrate (Connor et al., 2001). In vitro, PP1c alone dephosphorylates eIF2α and addition of recombinant R15A has no measurable effect under these conditions (Connor et al., 2001). Previously in the art, reconstituting PP1 holophosphatases has been challenging because regulatory subunits of phosphatases are natively unstructured (Peti et al., 2012). Here eIF2α holophosphatases with large fragments of the regulatory subunits (R15A325-636 and R15B340-698 hereafter referred to as R15A and R15B respectively) were reconstituted, these regulatory subunits are known to bind their respective inhibitors (Das et al., 2015; Tsaytler et al., 2011) (and WO2016162688A1 and WO2016162689A1) and recombinant PP1c.

Previous attempts in the art to reveal the activity of R15 inhibitors have failed, because the particular assay methodologies on which they relied did not depend only on the functioning of the regulatory subunits and a catalytic subunit (Choy et al., 2015; Crespillo-Casado et al., 2017). In one example, authors have used an R15 assay which depends on the presence of actin and did not reveal inhibition of R15 inhibitors (Crespillo-Casado et al., 2017). Knowing that R15s can be inhibited, the applicant reasoned that these proteins must have a positive function which ought to be inhibitable. It was established therefore that it is crucial to develop an assay to reveal and measure the positive function of R15s. No such assays have been reported or disclosed previously in the art which depend only and specifically on the catalytic and the regulatory subunits with no other co-factor, actin or else.

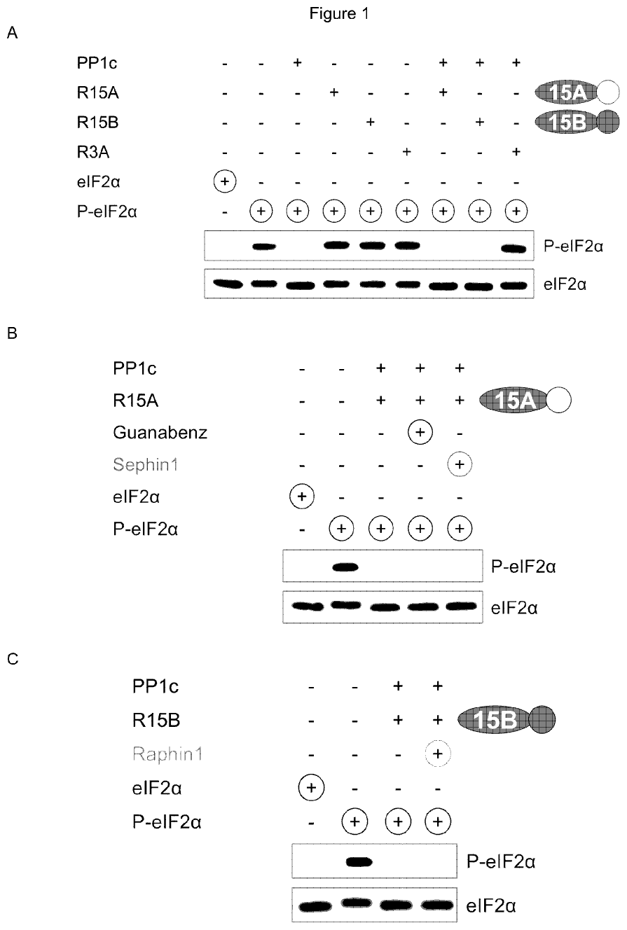

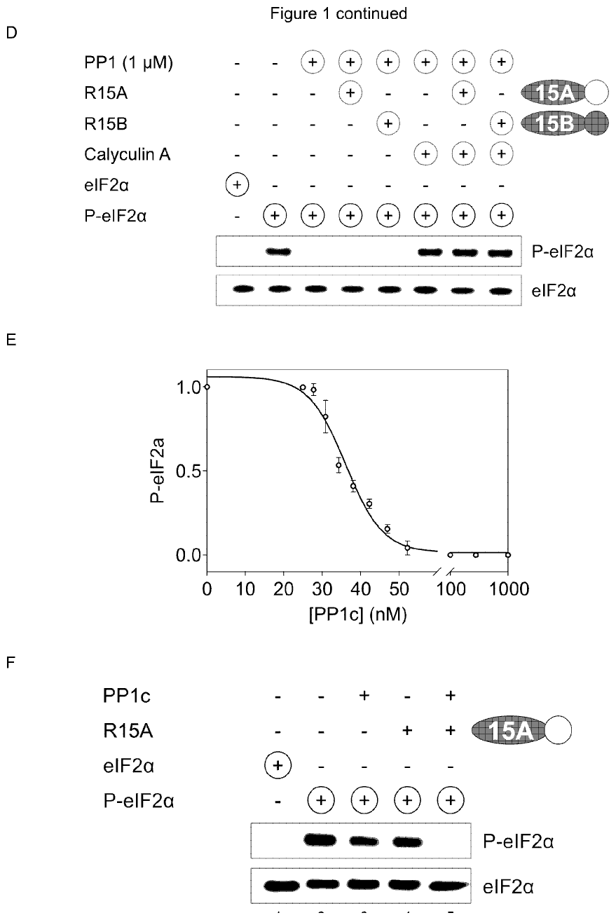

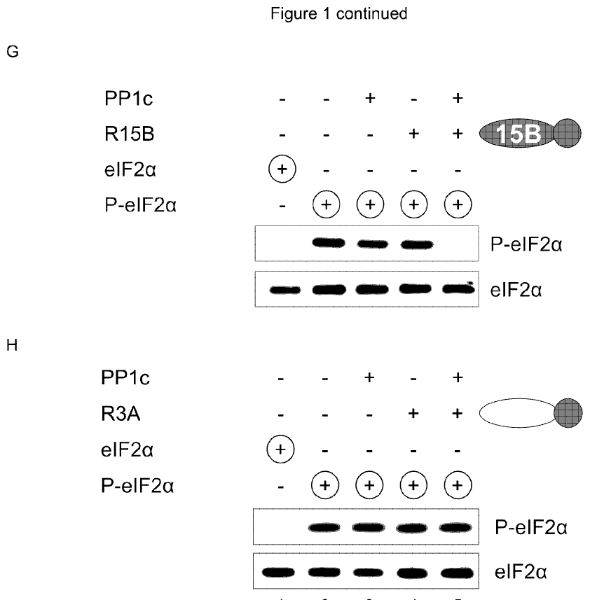

Commonly used phosphatase assays measure protein dephosphorylation using stoichiometric amounts of PP1c. Under such conditions, PP1c is not selective and dephosphorylates any substrates (Bollen et al., 2010). In the cells PP1c holophosphatases ought to be active at sub-stoichiometric concentrations (total PP1c concentration in cells is estimated to be 0.2 μM (Verbinnen et al., 2017)). Therefore titrations of PP1c were performed in order to elucidate its activity at a range of concentrations. These demonstrated that at physiological concentrations, PP1c alone is inactive, but becomes a proficient eIF2α phosphatase on the addition of R15A or R15B (FIGS. 1D to 1F). This creates a biochemically defined assay with minimal components (R15 and PP1) that recapitulates the physiological function of the holophosphatase.

The results obtained here reconcile the fundamental aspects of PP1c activity: in a non-physiological paradigm, using a high concentration of isolated PP1c, the enzyme is not selective. However, at low concentrations, PP1c alone is inactive but dephosphorylates a specific phospho-substrate when bound to a cognate regulatory subunit. This demonstrates that R15s and PP1c are necessary and sufficient components of the eIF2α holophosphatases. This is novel because (Chambers et al., 2015; Chen et al., 2015; Crespillo-Casado et al., 2017) have claimed that actin is required for R15 function.

The recombinant system developed here has the unique property of reporting on the biological activity of holophosphatases with minimal components: the catalytic subunit, the regulatory subunit and the substrate. It reveals a positive function for the regulatory R15 subunits, which is to convert an inactive PP1c into a holophosphatase capable of dephosphorylating eIF2α.

Having reconstructed functional R15 holophosphatases whose activity depends on the regulatory subunits, the molecular basis for their activities was next investigated. In this regard it was necessary to define the domains of regulatory subunits required for holophosphatase activity.

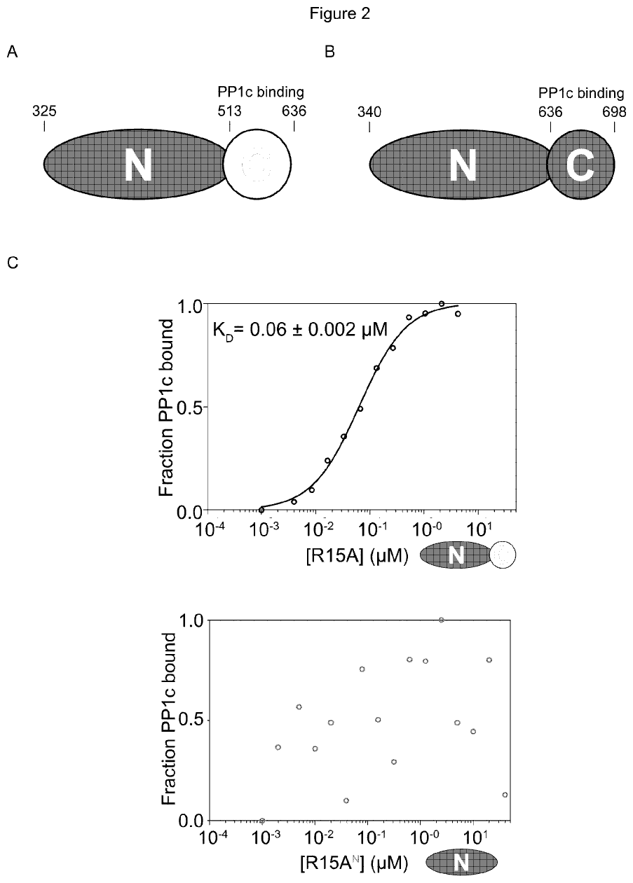

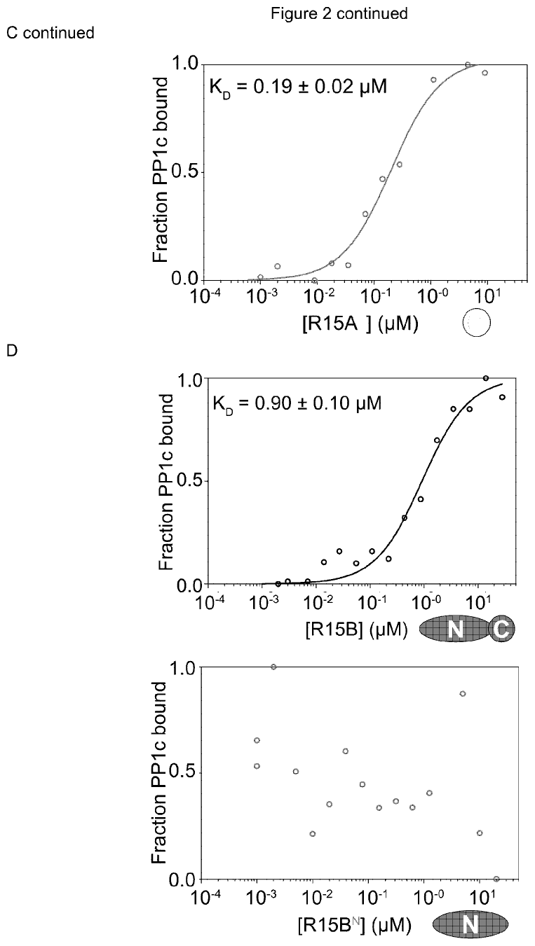

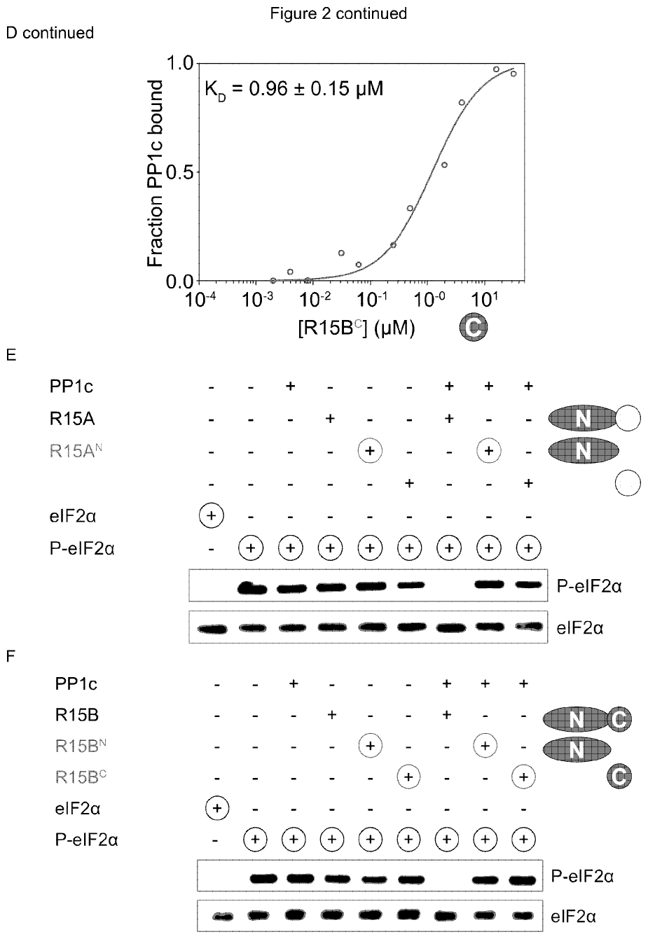

R15A and R15B contain a homologous PP1c binding region in their carboxy-terminal region and have divergent amino-terminal regions of unknown function (FIG. 2A). Thus, the recombinant and functional holophosphatases prepared were first characterized using thermophoresis affinity measurements and holophosphatase assays.

The thermophoresis affinity measurements demonstrated that the binding of the R15s to PP1c is almost entirely mediated by the carboxy terminal regions of the R15s. This was unexpected because previous work had shown that both the carboxy and the amino terminal region of R15A were required to bind the substrate (Choy et al., 2015; Rojas et al., 2015).

Interestingly, the affinity of R15B for PP1c was lower than that of R15A (FIG. 2B, C). Because R15A is inducible (Novoa et al., 2001), it has to compete with existing holophosphatases to recruit PP1c. The measured higher affinity of R15A for PP1c, relative to R15B, explains how the inducible R15A competes with R15B to recruit PP1c.

The PP1c binding region of regulatory subunits have been well characterized (Heroes et al., 2012) but the function(s) of the other domains are unclear. The recombinant system described herein enabled the contributions of the different domains of R15s to the activity of the eIF2α holophosphatases to be elucidated for the first time. Whilst the R15A^{C} carboxy-terminal fragment had a similar affinity to PP1c than the functional R15A (FIG. 2B), it was unable to convert PP1c into an active holophosphatase (FIG. 2D). Likewise, the carboxy-terminal fragment of R15B (R15B^{C}) was fully competent for recruiting PP1c (FIG. 2C) but was inactive in the phosphatase assay (FIG. 2E). This shows that in addition to the carboxy-terminal regions of R15s, which are required to bind PP1c, the amino-terminal regions of R15s are essential for their activity. This establishes a paradigm to study functional PP1c holophosphatases where the activity of the holophosphatase depends on a functional regulatory subunit.

Using a recombinant system containing two components, functional R15 holophosphatases and the substrate, the exquisite selective inhibition of R15s with Guanabenz, Sephin1 and Raphin1 has been recapitulated.

The present inventors have developed a completely recombinant system composed of two components, the R15-PP1c holophosphatase and the substrate, that faithfully recapitulates the selective and physiological function of holophosphatases. This unique feature of the assay is key to enabling the elucidation of the mechanism underlying the selective inhibition of the R15 inhibitors, and furthermore, functional studies of R15 holophosphatases. This assay is provides a method for selecting selective inhibitors of holophosphatases targeting their regulatory subunits

This method of study of functional and inhibitable recombinant R15 holophosphatases for the first time provides the mechanistic and functional explanation as to how a seemingly promiscuous enzyme is in fact highly selective. In vitro, free PP1c in isolation dephosphorylates any phospho-serine or -threonine protein as well as artificial substrates (Beullens et al., 1998). In cells, however, this does not occur because PP1c does not act solo but is bound to regulatory subunits. In this recombinant holophosphatase activity assay, it is shown that whilst PP1c alone can be an active phosphatase at high and non-physiological concentrations, there is a strict dependence on R15 regulatory subunits to dephosphorylate eIF2α at physiological concentrations, because R15-PP1c's have an increased affinity for eIF2α compared to PP1c alone. Importantly, an irrelevant regulatory subunit, R3A, decreased the affinity of PP1c to eIF2α, providing the molecular explanation for the substrate-specifier function of regulatory subunits. Knowing that PP1c cannot be detected alone in cells, but only as a complex with diverse regulatory subunits (Heroes et al., 2012) and that its total cellular concentration has been estimated to be ˜0.2 μM (Verbinnen et al., 2017), the notion that PP1c alone is inactive at low concentrations may represent a safeguard mechanism to ensure that PP1c remains inactive during its biogenesis, until bound to a regulatory subunit.

Another interesting aspect of R15 biology disclosed by the present inventors lies in the finding that R15A has a higher affinity for PP1c than does R15B (FIG. 2B, C). This makes sense knowing that R15A is inducible whilst R15B is constitutively expressed. The higher affinity of R15A for PP1c implies that R15A-PP1c may be the dominant eIF2α phosphatase during stress. This explains why R15B inhibitors induce a transient accumulation of eIF2α. Upon R15B inhibition, R15A is expressed through a negative feedback loop and becomes the dominant eIF2α phosphatase regulatory subunit, that ensures the reversibility of eIF2α phosphorylation.

Phosphatases have long been thought to be undruggable. The notion that holophosphatases can be selectively inhibited by targeting their regulatory subunits (Das et al., 2015; Tsaytler et al., 2011) has been challenged by unsuccessful attempts to reconstitute R15A inhibition in vitro with purified components (Choy et al., 2015; Crespillo-Casado et al., 2017). This only attests the previous challenge of studying holophosphatases, and can now be explained with the mechanistic insights into R15A holophosphatase function and inhibition provided by this invention. One laboratory used a short carboxy-terminal fragment of R15A constructs (Choy et al., 2015) that lacked the critical amino-terminal region, which herein is shown to be responsible for inhibition by Sephin1 and Guanabenz. Another previous report also failed to show the reconstitution of R15A inhibition by Sephin1. In the light of the present disclosure this is now understood to be because the in vitro assays used previously used ineffective methodologies, and therefore failed to reveal the activity of the selective R15A inhibitors.

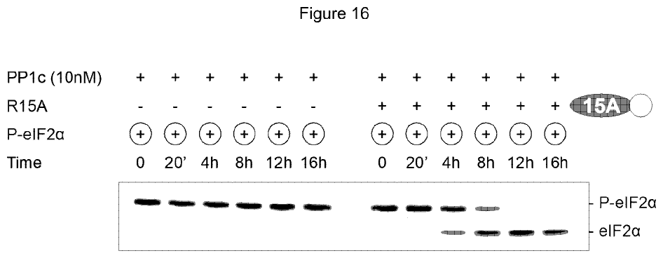

The present functional R15-PP1c holophosphatase also appears inactive in the conditions reported by (Crespillo-Casado et al., 2017), when the reaction is carried out for only 20 minutes, but the selection of a longer reaction time reveals its activity (FIG. 16). In addition, the recombinant proteins used here were different from those used in (Crespillo-Casado et al., 2017). Expression of functional PP1 in heterologous systems is notoriously difficult and the properties of native and recombinant PP1 often differ, with regard to selectivity, as well as sensitivity to inhibitors and regulatory subunits (Peti et al., 2013). Thus, the present inventors followed an optimized protocol to produce recombinant PP1 with nearly native properties (Peti et al., 2013) by co-expressing it in E. coli at 10° C. with the chaperones GroEL and GroES, in the presence of MnCl₂, which is known to stabilize the active site (Peti et al., 2013) (and Materials and Methods). In (Crespillo-Casado et al., 2017), PP1 was expressed at 18° C. without these chaperones. Moreover, the vitro assays in (Crespillo-Casado et al., 2017) relied on the presence of actin, yet the physiological relevance of actin for R15-holophosphatase function is unclear. Herein it is clearly established that actin is not required for the activity of R15 holoenzymes.

The conditions defined herein have established assays which depend on the regulatory subunits, this was the milestone required to elucidate the selective inhibition of the R15A holophosphatase with Sephin1 and Guanabenz, as well as the selective inhibition of the R15B holophosphatase by Raphin1.

The discovery of selective R15A inhibitors suggested the inhibitors did not disrupt the R15-PP1 binding interface for the following reasons. First, the regulatory subunit does not harbour catalytic function. Second, although it was observed that the R15A-PP1c complexes dissociate in cells treated with R15A inhibitor (Das et al., 2015; Tsaytler et al., 2011), it was suspected that this was a consequence of an conformational change in the holophosphatase rather than the result of the disruption of the R15A-PP1c interaction interface by the small molecule inhibitors for two reasons. First, the inhibitors are very small and therefore unlikely to disrupt the large R15A-PP1c interface (Choy et al., 2015). Moreover, this region is conserved in different regulatory subunits, so small molecules disrupting the conserved PP1c binding region will not be selective. The assays disclosed herein were used to uncover the molecular basis of the selective inhibition of R15 inhibitors. It was found that selective R15A inhibitors induce a conformational change in R15A but not in R15B. Conversely, Raphin1, a selective R15B inhibitor, induces a conformational change in R15B but not in R15A. The selective inhibition could be transferred from R15A to R15B and vice-versa by swapping their amino-terminal regions, indicating that the amino-terminal region of R15A is responsible for inhibition by Guanabenz and Sephin1, whilst the amino-terminal region of R15B is targeted by Raphin1. It was shown that the function of the amino-terminal region of R15s is to recruit the substrate and this function is inhibited by R15 inhibitors. This establishes that Guanabenz, Sephin1 and Raphin1 are selective inhibitors of R15s, which bind selectively to the amino-terminal region of their respective target, inducing a conformational change and inhibiting substrate recruitment.

Other inhibitors targeting the regulatory subunit could be identified using the same method and have different effect such as but not limited to affecting the binding to the catalytic subunit.

The power, the safety and the therapeutic benefit of selective inhibition of holophosphatases in neurodegenerative disease models have been previously exemplified by the present inventors. The suite of versatile assays described herein are generically applicable to hundreds of holophosphatases, providing access to an untapped class of enzyme, opening up a broad range of possibilities to manipulate cell function, perhaps for therapeutic benefit.

The methods and assays provided herein enable for the first time selection and characterization of selective serine/threonine holophosphatase inhibitors and there is a practical application for such methodology in a platform for permitting targeted drug screening in a previously uncharted area.

### Materials and Methods

### Protein Expression and Purification

The cDNA encoding human PPP1R15A, PPP1R15B and PPP1R3A regulatory subunits were cloned into a pMAL-c5×-His vector, encoding for an amino-terminal MBP-tag and a carboxy-terminal His₆-tag. The following constructs were cloned: PPP1R15A amino acids 325-636 (R15A), 325-512 (R15A^{N}), and 513-636 (R15A^{C}); PPP1R15B amino acids 340-698 (R15B), 340-635 (R15B^{N}), and 636-698 (R15B^{C}); and PPP1R3A amino acids 1-240 (R3A). R15A^{N}/R15B^{C} Chimeras were obtained by swapping the amino-terminal regions (R15^{N}) of R15A and R15B proteins described above, whilst maintaining the same carboxy-terminal regions (R15^{C}), to produce R15B^{N}/R15A^{C} and R15A^{N}/R15^{B} chimeras respectively, using In-Fusion cloning (Takara). All regulatory subunits were expressed in BL21 pLysS cells in Luria broth (LB) at 30° C. overnight. Proteins were purified by tandem affinity chromatography using HisTrap excel (GE Healtchare) followed by a MBPTrap HP column (GE Healthcare) using buffer A (50 mM Tris (pH 7.4), 200 mM NaCl). R15 proteins were stored at −80° C. and used within one month.

The cDNA encoding amino acids 7-330 of human PP1cα was cloned into a modified pGEX6p1 vector where the vector's GST-tag was replaced by an N-terminal Thio₆/His₆ tag (MGSDKIHHHHHH). PP1c^{D95A} mutant was obtained using site directed mutagenesis. PP1c proteins were expressed and purified using a protocol adapted from (Peti et al., 2013). PP1c proteins were expressed in BL21/pGro7 cells (Takara) in LB supplemented with 50 μg/ml Ampicillin, 35 μg/ml Chloramphenicol, and 2 mM MnCl₂. Cells were grown at 35° C. until OD₆₀₀ 0.5. Expression of the pGro7 plasmid was then induced with 1 g/L L-Arabinose and the temperature was immediately lowered to 10° C. At OD₆₀₀ 1.0, PP1c expression was induced with 0.1 mM IPTG. After 48 hours (h) expression at 10° C., cells were harvested and resuspended in fresh LB supplemented with 200 μg/ml Chloramphenicol and 2 mM MnCl₂. Cells were incubated for a further 2 h at 10° C. and then harvested. Thio₆/His₆-PP1c was purified by affinity chromatography on a HisTrap excel column (GE Healthcare), followed by size exclusion chromatography on a HiLoad 16/600 Superdex 200 pg column (GE Healthcare) using PP1c buffer (50 mM Tris (pH 7.4), 1 M NaCl, 2 mM MnCl₂). Purified PP1c was stored at −80° C., in PP1c buffer, in stocks above 20 μM, and diluted in the appropriate buffer immediately prior to use.

GST-tagged (amino-terminal) murine PERK kinase domain (amino acids 537-1114) (Addgene #21817) and His₆-tagged (carboxy-terminal) human eIF2α (amino acids 1-185) solubility mutant (Ito et al., 2004) were expressed in BL21 pLysS cells in LB at 37° C. for 6 h. Glutathione (GST)-PERK was purified on GST-sepharose beads (GE Healthcare) followed by size exclusion chromatography on a HiLoad 16/600 Superdex 200 pg column (GE Healthcare) using kinase buffer (50 mM Tris (pH 7.4), 100 mM NaCl, 10 mM MgCl₂, 5 mM DTT). eIF2α was purified by affinity chromatography on a HisTrap excel column (GE Healthcare), followed by size exclusion chromatography on a HiLoad 16/600 Superdex 200 pg column (GE Healthcare) using buffer A.

eIF2 Phosphorylation

eIF2α was phosphorylated on residue Ser51 using PERK kinase. 1 mg of purified PERK was incubated with GST-sepharose beads (GE Healthcare), pre-equilibrated with kinase buffer, for 30 minutes (′) at room temperature (RT). Excess PERK was removed by washing the beads 3 times with 1 ml kinase buffer. 500 μg of purified eIF2α, pre-dialysed in kinase buffer, was added to PERK-containing GST-beads. 5 mM ATP was added to the reaction and phosphorylation was allowed to proceed for 1 h at 37° C. The supernatant was collected and the phosphorylated eIF2α was further purified by size exclusion on a HiLoad 16/600 Superdex 200 pg column (GE Healthcare) using dephosphorylation buffer (50 mM Tris (pH 7.4), 1.5 mM EGTA (pH 8.0), 2 mM MnCl₂).

### Phosphorylase b Phosphorylation.

Phosphorylase b was phosphorylated using radioactive P33 isotope, following a protocol adapted from 4. 10 mg of Phosphorylase b (Sigma) was dissolved in 500 μl of reaction buffer (200 mM Tris [pH 7.4], 200 mM Glycerol-1-Phosphate, 200 μM CaCl2, 20 mM Mg(C2H3O2)2). 3.6 mg of Phosphorylase Kinase (Sigma) was dissolved in 1.8 ml of reaction buffer. 87.5 μl of Phosphorylase b solution and 125 μl of Phosphorylase Kinase were made up to 245 μl with reaction buffer. After 10 min of gentle shaking at RT, the solution was centrifuged at 15000 g for 5 min to remove protein precipitates. The supernatant was transferred to a new tube. The phosphorylation reaction was initiated by the addition of 1 μl of 10 mM ATP and 4 μl of [γ-33P]-ATP 10 mCi/ml stock (NEG602H100UC) (Perkin Elmer) and allowed to proceed for 2 h at 30° C. with shaking at 350 rpm. To stop the reaction, the solution was added directly to a PD MiniTrap G-25 desalting column (GE Healthcare), which was pre-equilibrated with dephosphorylation buffer (50 mM Tris [pH 7.4], 1.5 mM EGTA [pH 8.0], 2 mM MnCl2), using the gravity protocol. Desalted protein samples were collected and a Bradford assay was used to measure the concentrations of protein. Phosphorylated Phosphorylase b is known as Phosphorylase a.

In vitro dephosphorylation of P-eIF2α and ³³P Phosphorylase a.

PP1c was diluted to the appropriate concentration (as indicated in the figure legends) in dephosphorylation buffer immediately prior to use. Dephosphorylation reactions were carried out by pre-incubating diluted PP1c in the presence or absence of regulatory subunits (either 100 μM or 50 μM), and/or compounds (all at 100 μM, except for Salubrinal which was used at 30 μM), for 15′ at RT. All compounds were diluted in DMSO, and DMSO vehicle was used in all control experiments.

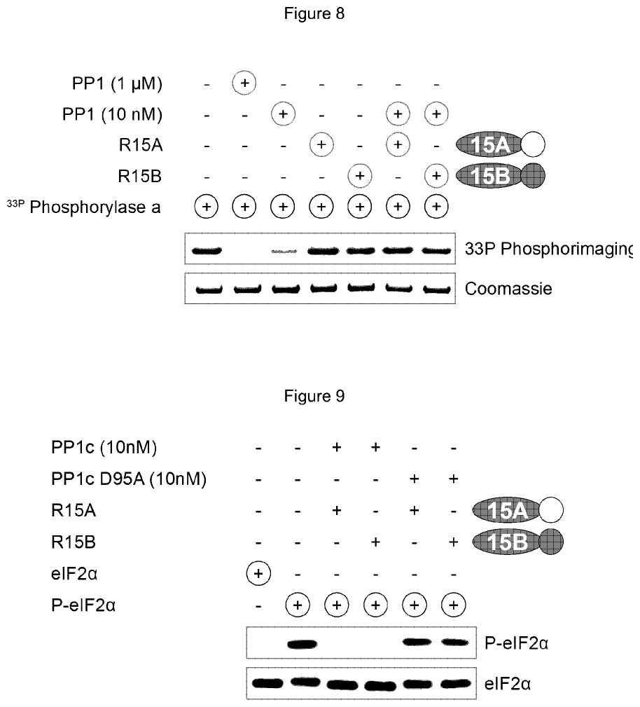

The reaction was initiated by the addition of 1 μM P-eIF2α or ³³P Phosphorylase a substrates and then incubated at 30° C. for 16 h with shaking at 350 rpm. Reactions were stopped by addition of 4× Laemmli sample buffer. eIF2α samples were analyzed by immunoblotting or Phos-tag gels. P-eIF2α dephosphorylation was analysed by immunoblotting using anti-Phospho-eIF2α (SerS1) (#9721) (Cell Signaling) and anti-eIF2α (ab26197) (AbCam) antibodies. Samples in FIG. 8 and FIG. 16 were run on a 15% SuperSep Phos-tag acrylamide gel (Alpha Laboratories) and visualized by staining with InstantBlue Protein Stain.

³³P Phosphorylase a samples were analyzed by phosphorimaging. 10 μl of samples were run on 4-12% NuPAGE Bis-Tris gels (Life Technologies) and visualized with InstantBlue Coomassie Protein Stain (Expedeon) to monitor total Phosphorylase levels. To measure levels of phosphorylated 33P Phosphorylase a, the gel was analyzed by phosphorimaging.

### In Vitro Dephosphorylation Assays Using Stoichiometric Levels of PP1c

As previously reported (Connor et al., 2001), in vitro PP1c alone (1 μM) dephosphorylated eIF2α, and in vitro co-incubation with R15A had no effect in this assay (FIG. 1A). R15B was also similarly inactive in this assay (FIG. 1A). As predicted previously (Hendrickx:2009hs; Connor et al., 2001), an unrelated regulatory subunit, R3A (glycogen-targeting subunit of protein phosphatase 1 (GM)), inhibited the dephosphorylation of eIF2α by PP1c (FIG. 1A). In such an assay, neither the selective R15A inhibitors, Guanabenz and Sephin1, nor the selective inhibitor of R15B, Raphin1, had any measurable effects (FIG. 1B, 1C). Like in previous similar studies with R15A inhibitors (Choy et al., 2015; Crespillo-Casado et al., 2017)}, the present inventors have also failed to reveal the activity of R15 inhibitors using this particular assay methodology. This is not surprising given that this assay does not depend on the regulatory subunits, it therefore cannot reveal the activity of their inhibitors.

### In Vitro Dephosphorylation Assays Using PP1c Titrations

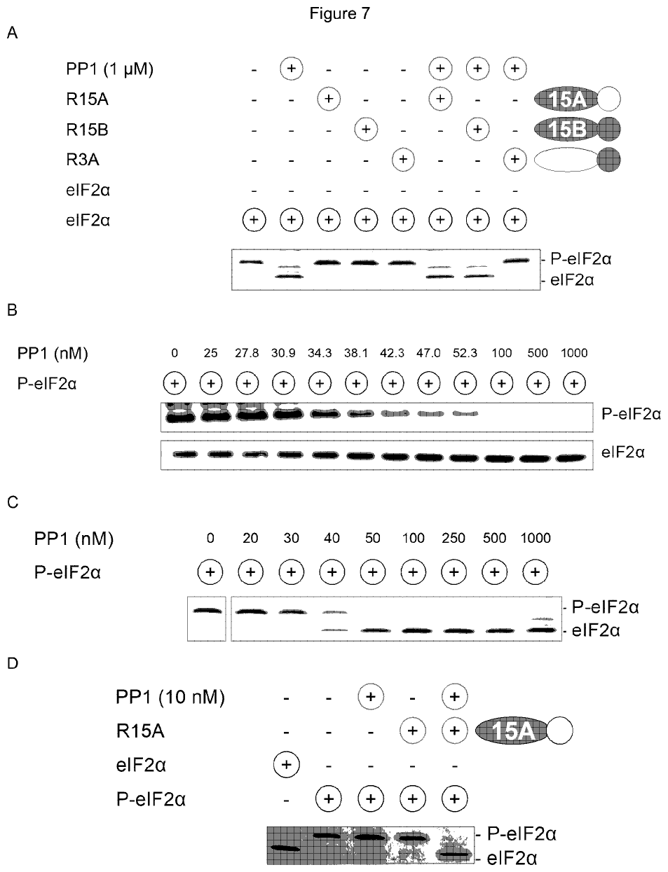

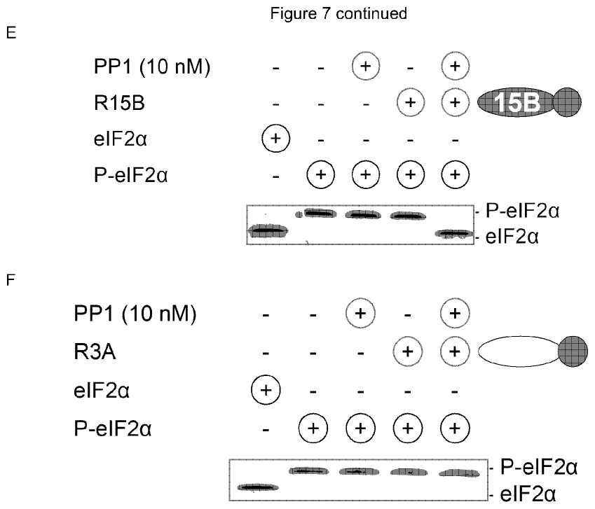

Titrations of PP1c showed that the enzyme was active at a stoichiometric concentration relative to the substrate (1 μM) but largely inactive at sub-stoichiometric concentrations (FIG. 1E and FIG. 7). Addition of R15A to a sub-stoichiometric concentration (10 nM) of PP1c enabled the complete dephosphorylation of eIF2α while PP1c (10 nM) alone had no effect (FIG. 1F). Likewise, addition of R15B, also converted the inactive PP1c (10 nM) into a proficient eIF2α phosphatase (FIG. 1G). Attesting the selectivity of R15s, addition of an unrelated regulatory subunit, R3A did not enable eIF2α dephosphorylation by PP1c (10 nM) (FIG. 1H and FIG. 7F). Conversely, the reconstituted R15 holoenzymes did not dephosphorylate an irrelevant substrate, Phosphorylase a, further confirming the selectivity of the holoenzymes as well as the selectivity of the assay (FIG. 8).

### Protein Binding to Biotinylated Compounds

Purified R15A protein was diluted to 1 μM in IP buffer (50 mM Tris [pH 7.4], 150 mM NaCl, 0.1% Tween20, 10% glycerol). 1 μM R15A, in 100 μl volume, was pre-cleared with 25 μl of Pierce Streptavidin Magnetic Beads (ThermoFisher) for 1 h at 4° C. with rotation at 20 rpm. The supernatants were collected and incubated with 0.5 mM biotinylated Guanabenz, Sephin1 or biotin control plus 25 μl of pre-equilibrated Pierce Streptavidin Magnetic Beads. Samples were incubated for 3 h at 4° C. with rotation at 20 rpm. The supernatant was removed, and samples were thoroughly washed and transferred to a fresh Eppendorf. The beads were washed thoroughly with 5×1 ml interaction buffer, with 30 min incubation each time, and then resuspended with 50 μl of 4% SDS Laemmli sample buffer. Samples were run on 4-12% NuPAGE Bis-Tris gels (Life Technologies), and analysed by immunoblotting using α-MBP HRP (E8038) (NEB) (to reveal R15A). Elution experiments were performed on beads containing R15A bound to biotinylated-Guanabenz or biotinylated-Sephin1, by adding 100 μl of interaction buffer containing 2 mM Guanabenz or Sephin1, respectively, or DMSO vehicle control. Samples were incubated for 10 min at 4° C. with 20 rpm rotation. 30 μl of supernatant was added to 10 μl of 16% SDS Laemmli sample buffer. 10 μl sample was run on 4-12% NuPAGE Bis-Tris gel (Life Technologies), and analysed by immunoblotting using α-MBP HRP (E8038) (NEB) (to reveal R15A).

### Characterization of R15s Using Thermophoresis Affinity Measurements and Phosphatase Assays

### Thermophoresis Affinity Measurements

Thermophoresis experiments were performed using a Monolith NT.115 instrument (NanoTemper Technologies). PP1c and P-eIF2α proteins were labeled using the Monolith NT Protein labelling Kit Red-NHS and stored for no longer than a month at −80° C. For thermophoresis, all protein dilutions were carried out in MST buffer (50 mM HEPES (pH 7.4), 100 mM NaCl, 0.1% Tween20, 2 mM MnCl₂). Labelled proteins (100 nM) were mixed with equal volumes of serial dilutions of the unlabelled binding partner. In holophosphatase binding experiments, labelled P-eIF2 was diluted to 100 nM in MST buffer containing 10 μM regulatory subunit and then mixed as above with titrations of unlabelled PP1c. All experiments were carried out in enhanced grade capillaries, using 100% LED power and 100% IR-laser (on for 25 seconds (″)) at 20° C. NanoTemper Analysis 1.2.101 software was used to fit the data with a nonlinear solution of the law of mass action and K_{D} values were determined. Each measurement was repeated in three independent experiments and K_{D} values were averaged. Standard error of the mean (SEM) values are shown.

R15A was found to bind PP1c with an affinity of 0.06 μM and this binding was largely encoded by the carboxy-terminal region of the protein, R15A^{C}, containing the PP1 binding site (FIG. 2B), as predicted previously (Connor et al., 2001; Novoa et al., 2001). The binding affinity of the functional R15A to PP1c was similar to that of a R15A552-567 peptide encoding the PP1c binding site (Choy et al., 2015; Crespillo-Casado et al., 2017). R15B also binds PP1c and this binding was also entirely mediated by the carboxy-terminal region of R15B (R15B^{C}; FIG. 2C). The affinity of R15B for PP1c was lower than that of R15A (FIG. 2B, C).

R15-PP1c Holophosphatases have a Higher Affinity for their Substrate than PP1c

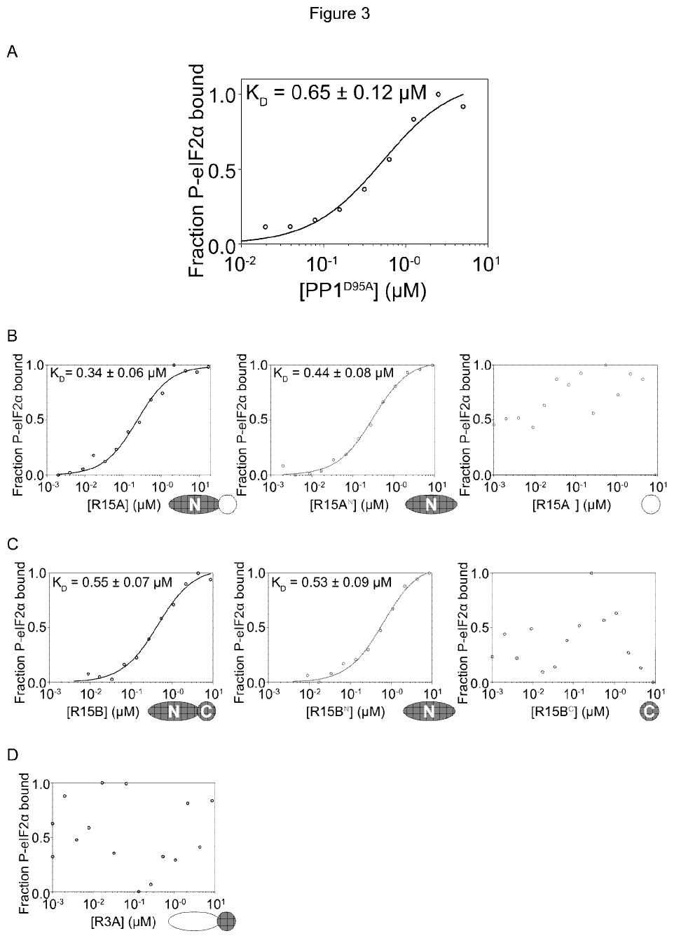

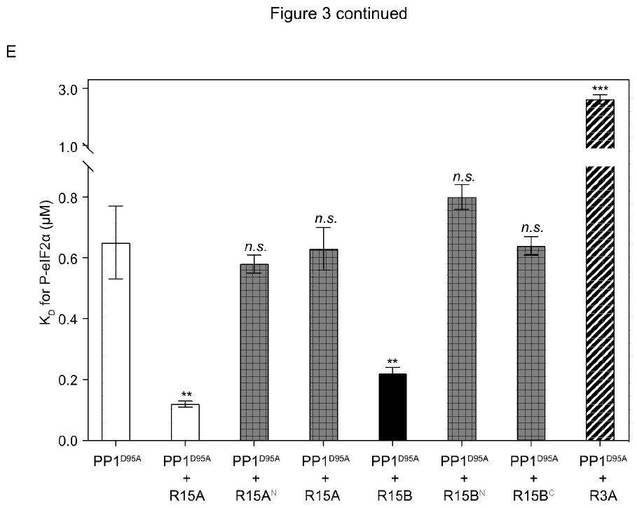

To gain further mechanistic insights into the activity of the functional eIF2α holophosphatases, the affinity of the different recombinant enzymes for their substrates was measured, using a mutant of PP1c (PP1c^{D95A}) with unaltered substrate binding but negligible activity (Zhang et al., 1996). As predicted previously (Zhang et al., 1996), PP1c^{D95A} was catalytically inactive (FIG. 9) but bound eIF2α with ˜0.65 μM affinity (FIG. 3A). Binding of R15s to eIF2α was next tested. Both R15A and R15B alone bound to eIF2α (FIG. 3A, B), confirming their function in recruiting the substrate. Because studies with contradictory results have been reported (Choy et al., 2015; Rojas et al., 2015), it is unclear which region of R15A binds eIF2α, and no such studies have previously been performed with R15B. Thus, the region of R15s which binds the substrate was investigated. No binding of the carboxy-terminal region of either R15 to eIF2α was detected, whilst the amino-terminal region alone of either R15 bound eIF2α, similar to the functional R15A and R15B (FIG. 3B). Thus, binding of R15A and R15B to eIF2α is encoded entirely by their amino-terminal regions (FIG. 3B). The affinities of the R15A-PP1c^{D95A} and R15B-PP1c^{D95A} for eIF2α (FIG. 3C and Table 1) were respectively 5.4 and 3.0 times higher than the affinity of the isolated PP1c^{D95A} (FIG. 3A). The higher affinities of the functional holophosphatases R15A-PP1c and R15B-PP1c for their cognate substrate, eIF2α, relative to the catalytic subunit alone, explains why R15A and R15B convert an inactive PP1c into a functional holophosphatase when using sub-stoichiometric concentrations of PP1c. When R15 holophosphatases were prepared with carboxy-terminal fragments of regulatory subunits the affinity of these complexes to eIF2α was as low as that of the isolated PP1c^{D95A} (FIG. 3C and Table 1). The failure of the carboxy-terminal fragments of R15s to increase the affinity of the R15C-PP1c complex to the substrate explains why the R15C-PP1c complexes were inactive (FIG. 2E, F). The amino-terminal fragments of R15 s didn't alter the binding affinity of PP1c^{D95A} to eIF2α (FIG. 3C and Table 1). An irrelevant regulatory subunit, R3A, had the opposite effect to the R15s and decreased PP1c^{D95A} affinity to eIF2α (FIG. 3C and Table 1). This defines the molecular basis for the dual functions of the regulatory subunits: R15A and R15B increase the affinity of PP1c to their cognate substrates whilst an irrelevant regulatory subunit, R3A, decreases the affinity of PPc1 to a non-cognate substrate. In addition, this demonstrates that both the eIF2α-binding amino-terminal region of R15 s and their PP1c-binding carboxy-terminal region are required for their function.

### Selective R15 Inhibitors Block Substrate Recruitment

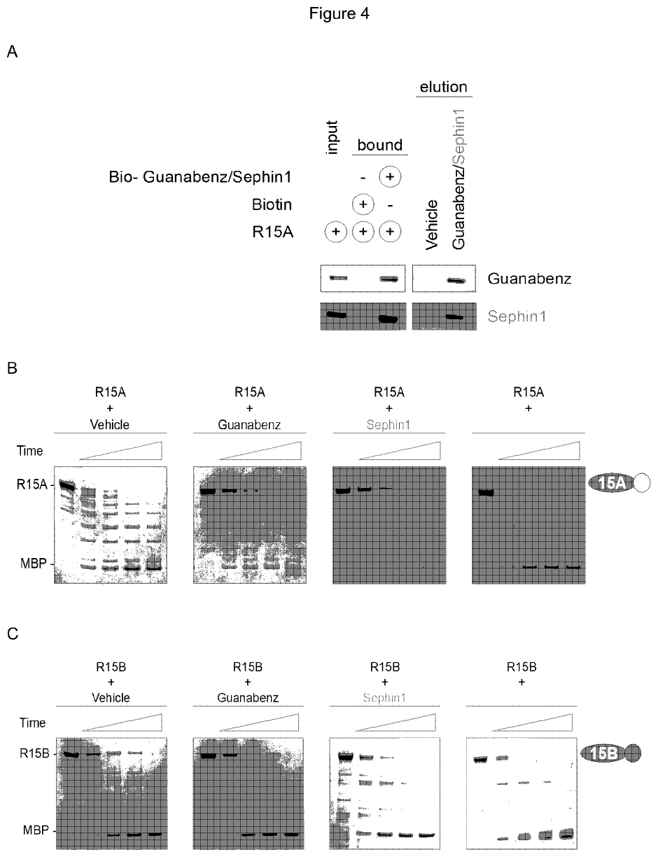

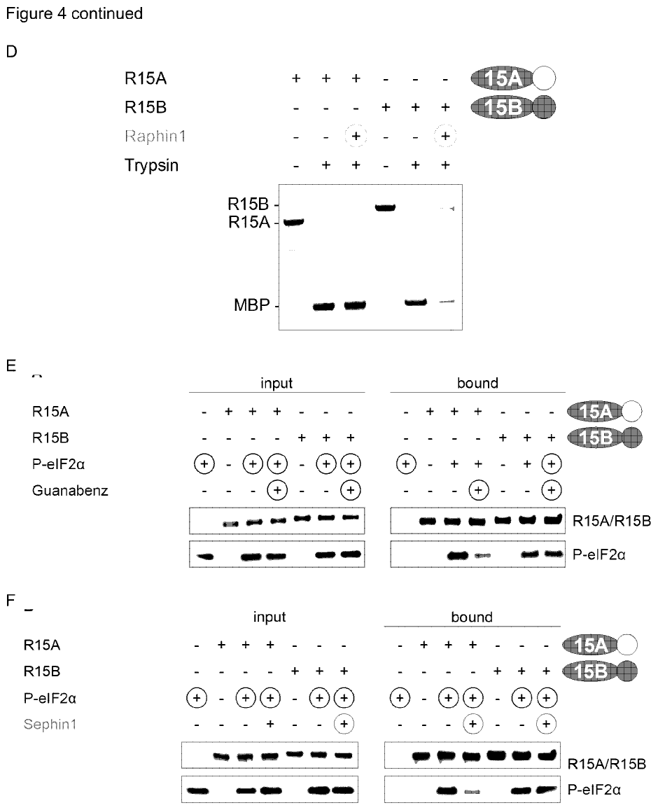

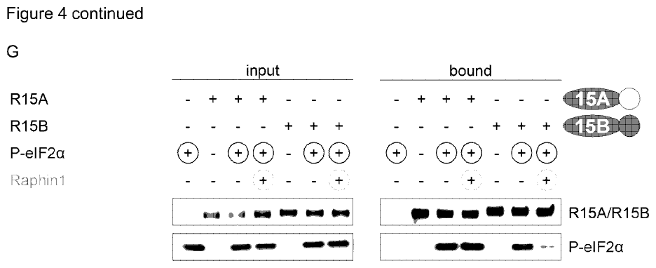

Having recapitulated the function of regulatory subunits of holophosphatases in a recombinant system, their inhibitors were evaluated. The present inventors confirmed that Guanabenz and Sephin1 directly bind R15A as previously reported (Das et al., 2015; Tsaytler et al., 2011) (FIG. 4A). Raphin1 selectively binds and inhibits R15B as disclosed in WO2016162688A1 and WO2016162689A1. Furthermore, it was observed that the binding of R15A to the selective inhibitors Guanabenz and Sephin1 was not covalent because R15A, immobilized on biotinylated-Guanabenz or biotinylated-Sephin1, was eluted with excess of Guanabenz or Sephin1 respectively (FIG. 4a).

The selectivity of the inhibitors suggested that they ought to target a divergent region of R15A and R15B. Having established that functional R15 holophosphatases have an increased affinity for eIF2α relative to the isolated PP1c and knowing that the functional R15s specifically bind their respective inhibitors (Das et al., 2015; Tsaytler et al., 2011) and (WO2016162688A1 and WO2016162689A1), whether the inhibitors altered substrate recruitment was tested. The inhibitors were unsuitable for thermophoresis experiments and thus, pull-down assays were performed. Guanabenz, Sephin1 and Raphin1 were synthesised as described in (Das et al., 2015; Tsaytler et al., 2011).

### Pull Down Experiments

Purified MBP-tagged regulatory subunits (200 nM), P-eIF2α (1 μM), PP1c (500 μM), and 200 μM compounds, or DMSO vehicle, were added as appropriate to 20 μl amylose magnetic bead, pre-equilibrated with interaction buffer (50 mM Tris (pH 7.4), 200 mM NaCl, 0.05% Tween20) in 200 μl volume. All protein dilutions were carried out in interaction buffer. 5% input sample was removed, and added to 4× Laemmli sample buffer for later analysis. The beads were incubated for 10′ at 4° C. The supernatant was removed, and the beads were washed thoroughly with interaction buffer and then resuspended with 50 μl of 2× Laemmli sample buffer. Samples were run on 4-12% NuPAGE Bis-Tris gels (Life Technologies) and analysed by immunoblotting, using anti-MBP HRP (E8038) (NEB), anti-eIF2α (ab26197) (AbCam) or anti-PPP1A (ab137512) (AbCam) antibodies.

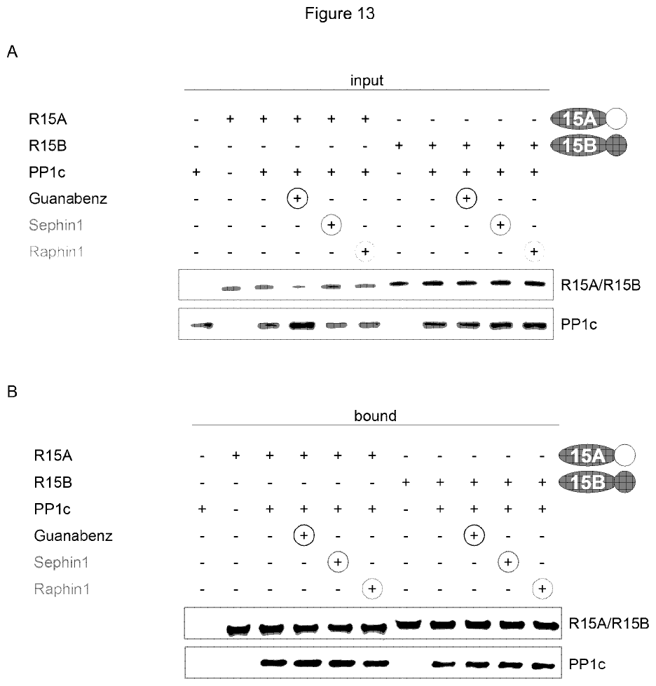

It was found that Guanabenz, a selective R15A inhibitor, decreased the binding of R15A to eIF2α (FIG. 4A, lane 10,11). This inhibition was selective because no such effect was observed with Guanabenz and R15B (FIG. 4A, lane 13,14). Likewise, Sephin1 also selectively prevented the binding of eIF2α to R15A (FIG. 4B, lane 10,11) but not R15B (FIG. 4B, lane 13,14). Unlike what has been observed in cells (Das et al., 2015; Tsaytler et al., 2011), dissociation of the recombinant holophosphatases upon treatment with their respective inhibitors was not observed (FIG. 13) suggesting that some cellular factors ought to be required for this dissociation. In contrast to Guanabenz and Sephin1, the selective R15B inhibitor Raphin1 decreased the binding of eIF2α to R15B (FIG. 4C, lane 13, 14) and this inhibition was not observed with R15A (FIG. 4C, lane 11,12). This confirmed the selectivity of the inhibitors and demonstrated that they compromise the ability of R15s to recruit the eIF2α substrate.

R15 Inhibitors Induce a Conformational Change in their Selective Target

To elucidate the mechanism by which inhibitors impair the holophosphatases, it was investigated whether the inhibitors induced a conformational change in their respective regulatory subunits. The experimental paradigm, using a mild trypsin proteolysis, previously employed to reveal a conformational change induced by a PTP1B inhibitor (Krishnan et al., 2014), was used. To assess whether binding of R15A inhibitors induced a conformational change in their target, the sensitivity of R15s to mild proteolysis in the presence or in absence of inhibitors was measured.

As a control, a close chemical derivative of Guanabenz, compound C3 ((E)-2-((3-chloropyridin-2-yl)methylene)hydrazine-1-carboximidamide) (1-[(E)-(3-chloro-2-pyridyl)methyleneamino]guanidine)), was synthesised as follows:

### Preparation of (E)-2-((3-chloropyridin-2-yl)methylene)hydrazine-1-carboximidamide

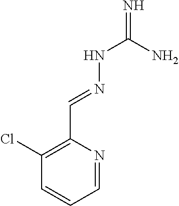 [SMILES: N=C(N)N/N=C/c1ncccc1Cl]

3-chloropyridine-2-carbaldehyde (0.25 g, 0.001773 Mol) was dissolved in ethanol (10 mL) at room temp. 1-aminoguanidine hydrochloride (0.196 g, 0.001773 Mol) and sodium acetate trihydrate (0.241 g, 0.001773 Mol) were added and the reaction was heated to reflux at 80° C. for 3 h. The reaction was dumped into a solution of saturated sodium bicarbonate. The solid was filtered off and the solid residue washed with demineralised water, hexane and ether. The solid was dried and triturated with diethyl ether. Product yield 0.170 g (0.00086 Mol, 48%)

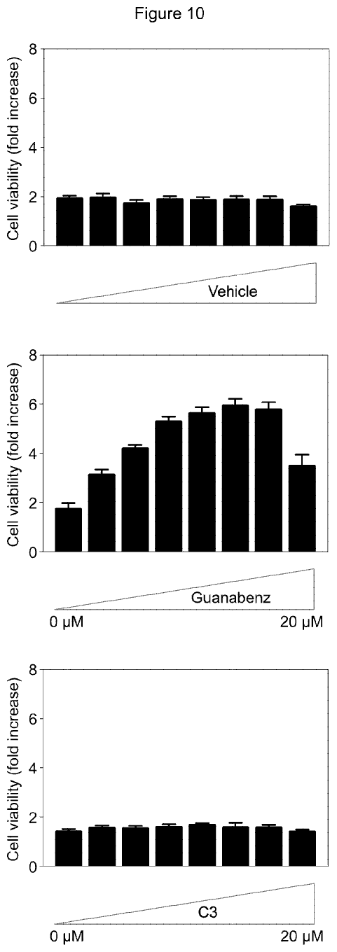

C3 was then utilized in the assays as described. C3, unlike Guanabenz, was found to be inactive in cytoprotection from ER stress (FIG. 10).

### Limited Trypsin Proteolysis (FIG. 4B, C)

Purified R15A, R15B or MBP were diluted to 0.5 μM in phosphate buffered saline (PBS) (13.7 mM NaCl, 0.27 mM KCl, 0.8 mM NaHPO4, 0.2 mM KH2PO4) [pH 7.4], in a final volume of 200 μl, and incubated for 15 min at room temperature with 100 μM compound, or DMSO vehicle. Reactions were initiated by addition of 2.5 nM of trypsin from bovine pancreas (Sigma), made from the lyophilised powder in PBS. Reactions were allowed to proceed at 22° C. with shaking at 350 rpm. At time points 30 min, 1 h, 2 h or 3 h, 30 μl of sample was removed from the mix and digestion was stopped by addition of 10 μl of 16% SDS Laemmli sample buffer. Samples were run on 4-12% NuPAGE Bis-Tris gels (Life Technologies). Proteins were visualised by staining with InstantBlue Coomassie Protein Stain (Expedeon) protein stain.

### Limited Trypsin Proteolysis (FIG. 4D)

Purified R15A and R15B were diluted to 3 μM in PBS, and incubated for 15′ at RT with 100 μM Raphin1, or DMSO vehicle. Reactions were initiated by addition of 5 nM of trypsin from bovine pancreas (Sigma), made up in PBS, and allowed to proceed for 5′ at 22° C., with shaking at 350 rpm. Digestion was stopped by addition of 4× Laemmli sample buffer and samples were run on 4-12% NuPAGE Bis-Tris gels (Life Technologies). Proteins were visualised by staining with InstantBlue Protein Stain.

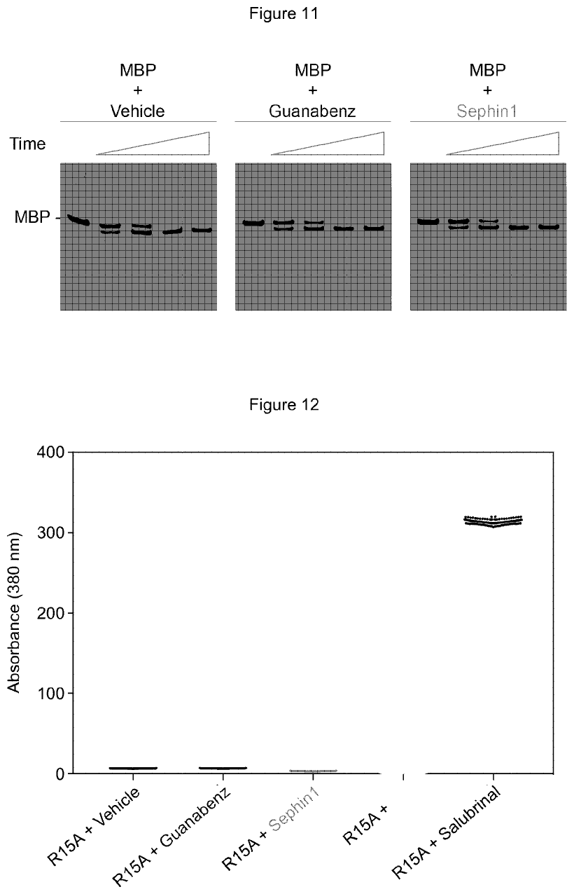

As expected for natively unstructured proteins (Beullens et al., 1998), R15A and R15B were sensitive to a mild trypsin proteolysis (FIG. 4 B,C). Addition of Guanabenz decreased the sensitivity of R15A to trypsin proteolysis (FIG. 4B). No such protective effects on the sensitivity to trypsin were seen when using Guanabenz and R15B (FIG. 4C), confirming the selectivity of Guanabenz for R15A. Similar results were obtained using Sephin1 (FIG. 4B,C). Importantly, the negative compound C3 did not alter the protease sensitivity of R15 to mild proteolytic treatment (FIG. 4B,C). Further attesting the specificity of the findings, the compounds had no measurable effects on the protease sensitivity of MBP showing that the compounds did not inhibit trypsin (FIG. 11). This also suggests that the compounds do not induce aggregation of proteins. The observation that the interaction between R15A and its inhibitors was reversible (FIG. 4A) also indicated that the compounds did not cause aggregation of the proteins. To formerly address this possibility, light scattering analysis was performed. No aggregation of R15A at a saturating concentration of Guanabenz, Sephin1 or C3 (1 mM) was found (FIG. 12). However, Salubrinal at 50 μM induced robust aggregation (FIG. 12). Together these results confirm that the R15A inhibitors Guanabenz and Sephin1 directly and reversibly bind R15A. The binding of Guanabenz and Sephin1 to R15A selectively alters the sensitivity to trypsin of R15A but not R15B, implying that they induce a conformational change only in their target.

Raphin1, the selective R15B inhibitor had no effect on the trypsin sensitivity of R15A but protected R15B (FIG. 4D). These results demonstrate that the selective inhibitors of R15s induce a conformational change in their respective target which decreases their sensitivity to proteolysis. This conformational change induced by the selective inhibitors may explain how they prevent the substrate recruitment function of R15s.

### The Selective Inhibition of R15 Inhibitors Recapitulated by the Recombinant System

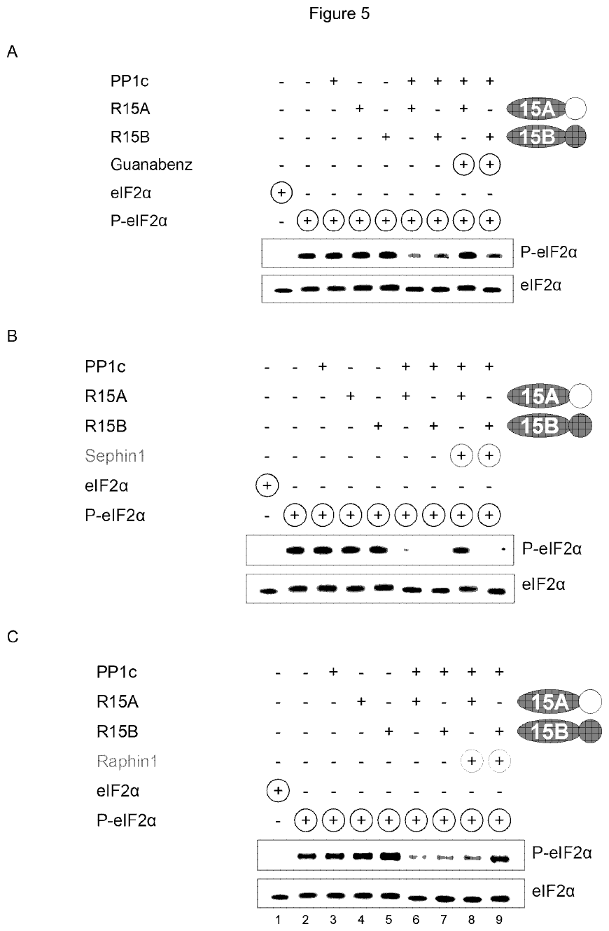

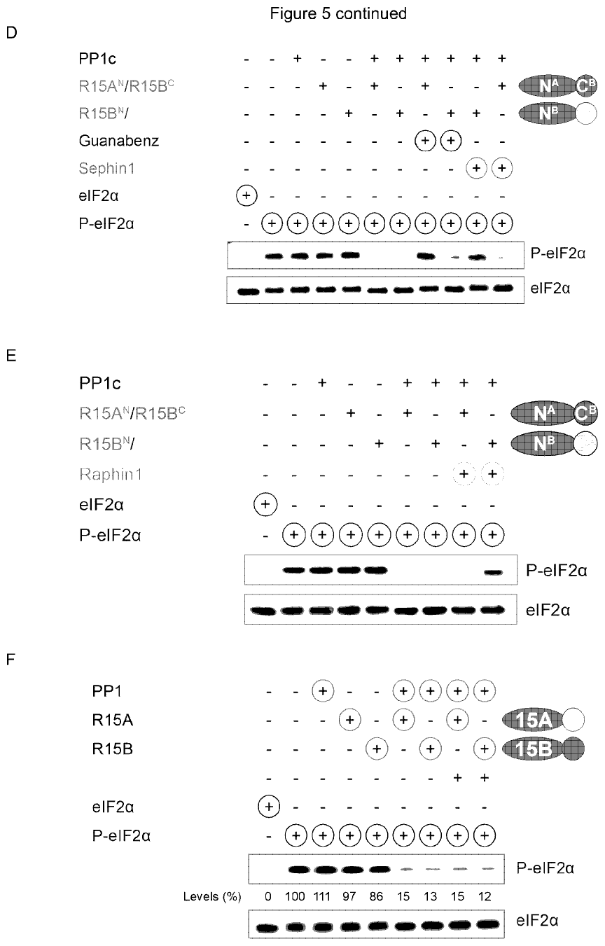

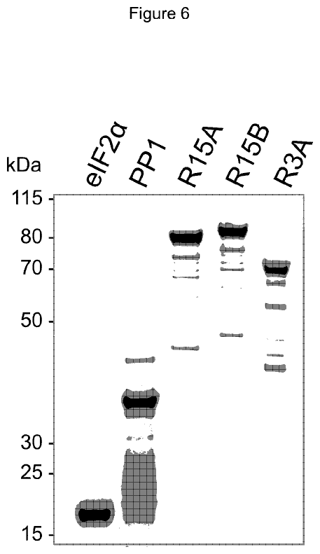

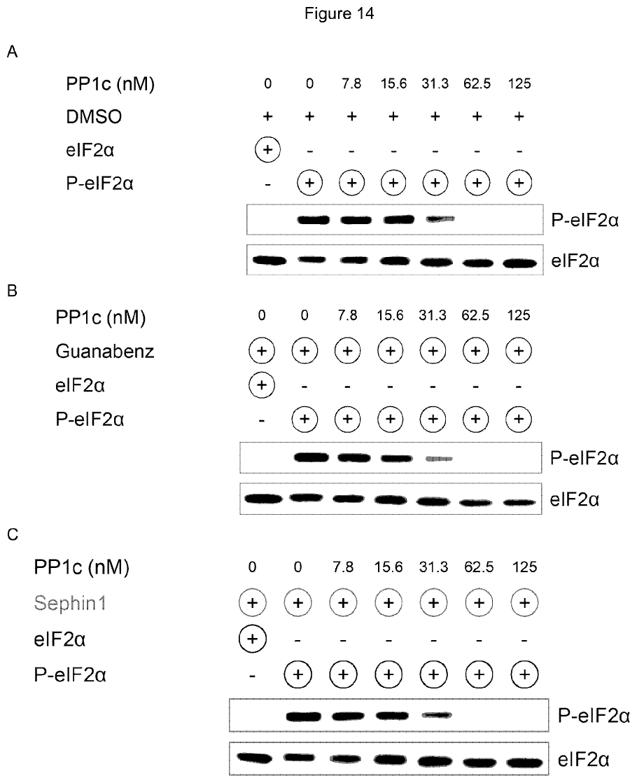

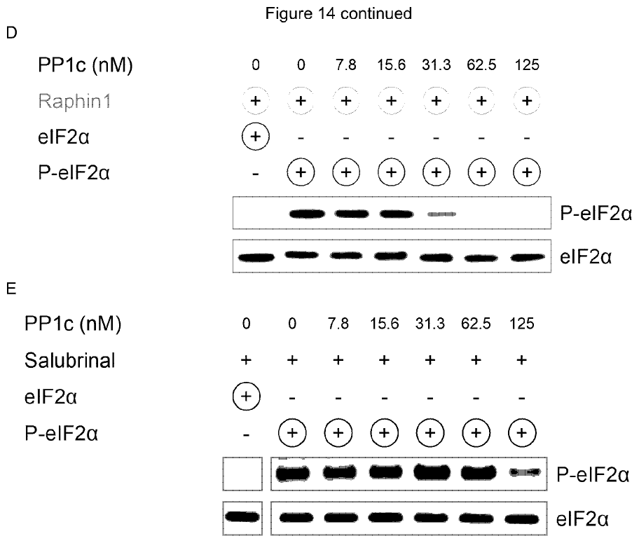

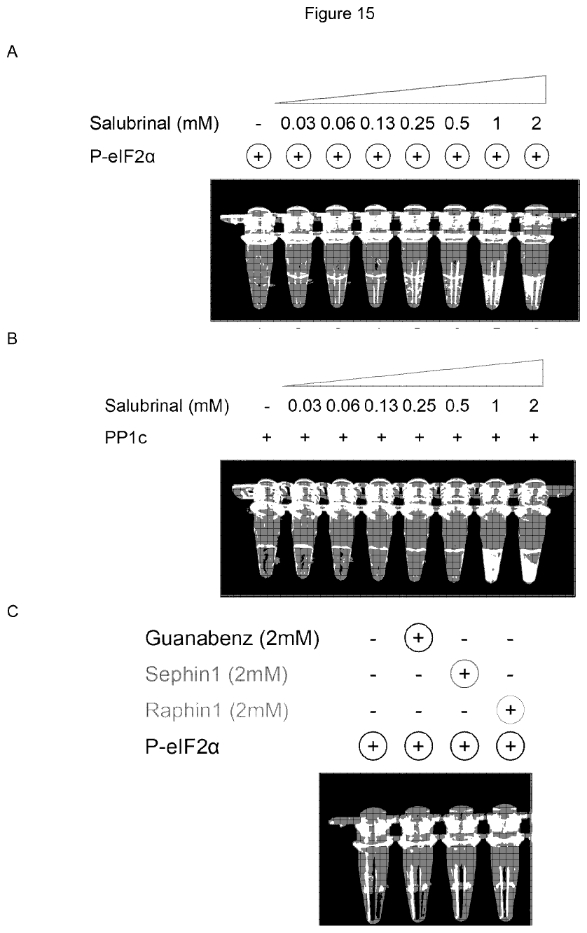

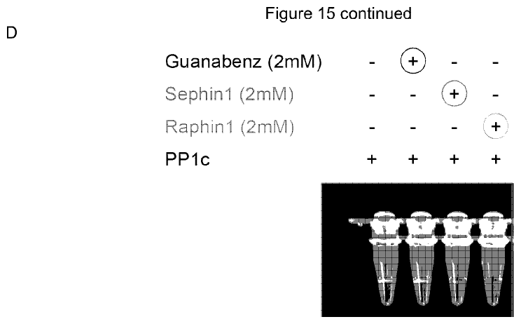

Whilst recombinant R15A-PP1c and R15B-PP1c were both active eIF2α holophosphatases, Guanabenz selectively inhibited R15A-PP1c but not R15B-PP1c as shown in in vitro dephosphorylation assays, as above (FIG. 5A, lane 8, 9). Sephin1 also selectively inhibited R15A-PP1c in the recombinant system (FIG. 5B, lane 8, 9). In contrast to Guanabenz and Sephin1, Raphin1 selectively inhibited R15B-PP1c but not R15A-PP1c (FIG. 5C, lane 8, 9). As observed before (Das et al., 2015; Tsaytler et al., 2011), Guanabenz, Sephin1 and Raphin1 (100 μM) did not inhibit PP1c (FIGS. 14 C and D). In contrast, it was found that Salubrinal inhibited PP1c at 100 μM (FIG. 14 E). This may not be selective, however, because Salubrinal induced the precipitation of proteins (FIGS. 15 A and B), unlike Guanabenz, Sephin1 and Raphin1 (FIGS. 15 C and D). Having found that the selective R15 inhibitors disrupted recruitment of eIF2α it was suspected that the inhibitors bound in the eIF2α-binding amino-terminal region (R15A^{N} or R15B^{N}) of their respective targets. The unstructured nature of R15s rendered mutagenesis studies inconclusive. Thus, a different approach of generating chimeras was adopted, by swapping the R15^{N} amino-terminal regions (R15A^{N} or R15B^{N}) to assess whether the sensitivity of the R15s to the selective inhibitors would be concomitantly swapped. Swapping the amino-terminal region of R15s generated the functional enzymes R15A^{N}/R15B^{C} and R15B^{N}/R15A^{C} (FIG. 5D, lanes 6, 7). The R15A^{N}/R15B^{C} chimera was inhibited by Guanabenz and Sephin1 (FIG. 5D, lane 8, 10). In contrast, R15B^{N}/R15A^{C} chimera was largely insensitive to Guanabenz and Sephin1 (FIG. 5D, lanes 9 and 11). The selective R15B inhibitor Raphin1 was next tested on the chimeras. It was found that Raphin1 selectively inhibited R15B^{N}/R15A^{C} (FIG. 5E, lane 9) but not R15A^{N}/R15B^{C} (FIG. 5E, lane 8). Confirming the selectivity of the assays and of the R15A inhibitors, compound C3 was inactive and did not inhibit R15A-PP1 or R15B-PP1 (FIG. 5F). This establishes that a defined recombinant system containing only three components, R15, PP1 and the eIF2α substrate, recapitulates the exquisitely selective inhibition of R15A by Guanabenz and Sephin1.

### Visualisation of Protein Precipitates

P-eIF2α (1 μM) or PP1c (1 μM) were diluted with dephosphorylation buffer, and compounds were added, at the indicated concentration. Samples were allowed to equilibrate for 5′ at RT before visualising.

### Light Scattering.

Light scattering experiments, to monitor the presence of protein aggregates, were performed with a Varian Cary Eclipse Fluorescence Spectrophometer (Agilent), as used in (Wilcken et al., 2012). R15A was diluted to 5 μM, and 1 mM Guanabenz, 1 mM Sephin1, 1 mM C3, 50 μM Salubrinal or DMSO vehicle control were added. Samples were incubated for 10 min at RT. In a range of 320-400 nm, soluble proteins do not absorb, whereas protein aggregates do. Therefore, light scattering was measured at 380 nm emission, 380 nm absorption to monitor the presence of aggregates. For each sample, 100 data points were collected over a period of 10 min, at 20° C., with constant stirring.

### Cytoprotection Experiments

HeLa cells (40,000 cells/ml) were plated in a 96-well plate and treated with different concentrations of compound, as indicated, or DMSO vehicle in the presence of 250 ng/ml Tunicamycin for 72 h. To monitor cell death, 1/2000 dilution of the CellTox green dye (Promega) was added to the media. The growth of the cells was monitored over time and pictures taken every 2 h with the IncuCyte ZOOM system and analysed by the IncuCyte ZOOM software (Essen BioScience). To compare different compounds and their cytoprotective effect, a growth ratio for each time point was calculated:

Growth ratio=(Phase confluency (%) at X hours)/(Phase confluency (%) at 0 hours)

The end point of the assay (72 h) was chosen for generating the graphs.

### REFERENCES

Beullens, M., Stalmans, W., and Bollen, M. (1998). The biochemical identification and characterization of new species of protein phosphatase 1. Methods Mol. Biol. 93, 145-155.
Bollen, M., Peti, W., Ragusa, M. J., and Beullens, M. (2010). The extended PP1 toolkit: designed to create specificity. Trends Biochem. Sci. 35, 450-458.
Chambers, J. E., Dalton, L. E., Clarke, H. J., Malzer, E., Dominicus, C. S., Patel, V., Moorhead, G., Ron, D., and Marciniak, S. J. (2015). Actin dynamics tune the integrated stress response by regulating eukaryotic initiation factor 2α dephosphorylation. eLife 4.
Chen, R., Rato, C., Yan, Y., Crespillo-Casado, A., Clarke, H. J., Harding, H. P., Marciniak, S. J., Read, R. J., Ron, D., and Nelson, W. J. (2015). G-actin provides substrate-specificity to eukaryotic initiation factor 2α holophosphatases. eLife 4, e04871.
Choy, M. S., Page, R., and Peti, W. (2012). Regulation of protein phosphatase 1 by intrinsically disordered proteins. Biochem. Soc. Trans. 40, 969-974.
Choy, M. S., Yusoff, P., Lee, I. C., Newton, J. C., Goh, C. W., Page, R., Shenolikar, S., and Peti, W. (2015). Structural and Functional Analysis of the GADD34: PP1 eIF2α Phosphatase. Cell Reports 11, 1885-1891.
Connor, J. H., Weiser, D. C., Li, S., Hallenbeck, J. M., and Shenolikar, S. (2001). Growth arrest and DNA damage-inducible protein GADD34 assembles a novel signaling complex containing protein phosphatase 1 and inhibitor 1. Mol. Cell. Biol. 21, 6841-6850.
Crespillo-Casado, A., Chambers, J. E., Fischer, P. M., Marciniak, S. J., and Ron, D. (2017). PPP1R15A-mediated dephosphorylation of eIF2α is unaffected by Sephin1 or Guanabenz. eLife 6, e26109.
Das, I., Krzyzosiak, A., Schneider, K., Wrabetz, L., D'Antonio, M., Barry, N., Sigurdardottir, A., and Bertolotti, A. (2015). Preventing proteostasis diseases by selective inhibition of a phosphatase regulatory subunit. Science 348, 239-242.
Hendrickx, A., Beullens, M., Ceulemans, H., Abt, Den, T., Van Eynde, A., Nicolaescu, E., Lesage, B., and Bollen, M. (2009). Docking motif-guided mapping of the interactome of protein phosphatase-1. Chem. Biol. 16, 365-371.
Heroes, E., Lesage, B., Gornemann, J., Beullens, M., Van Meervelt, L., and Bollen, M. (2012). The PP1 binding code: a molecular-lego strategy that governs specificity. FEBS Journal 280, 584-595.
Ito, T., Marintchev, A., and Wagner, G. (2004). Solution structure of human initiation factor eIF2alpha reveals homology to the elongation factor eEF1B. Structure 12, 1693-1704.
Krishnan, N., Koveal, D., Miller, D. H., Xue, B., Akshinthala, S. D., Kragelj, J., Jensen, M. R., Gauss, C.-M., Page, R., Blackledge, M., et al. (2014). Targeting the disordered C terminus of PTP1B with an allosteric inhibitor. Nature Chemical Biology 10, 558-566.
Novoa, I., Zeng, H., Harding, H. P., and Ron, D. (2001). Feedback inhibition of the unfolded protein response by GADD34-mediated dephosphorylation of eIF2alpha. The Journal of Cell Biology 153, 1011-22.
Peti, W., Nairn, A. C., and Page, R. (2012). Folding of Intrinsically Disordered Protein Phosphatase 1 Regulatory Proteins. Curr Phys Chem 2, 107-114.
Peti, W., Nairn, A. C., and Page, R. (2013). Structural basis for protein phosphatase 1 regulation and specificity. Febs J. 280, 596-611.
Rojas, M., Vasconcelos, G., and Dever, T. E. (2015). An eIF2α-binding motif in protein phosphatase 1 subunit GADD34 and its viral orthologs is required to promote dephosphorylation of eIF2α. Proc. Natl. Acad. Sci. U.S.A. 201501557-14.
Tsaytler, P., Harding, H. P., Ron, D., and Bertolotti, A. (2011). Selective inhibition of a regulatory subunit of protein phosphatase 1 restores proteostasis. Science 332, 91-94.
Verbinnen, I., Ferreira, M., and Bollen, M. (2017). Biogenesis and activity regulation of protein phosphatase 1. Biochem. Soc. Trans. 45, 89-99.
Wilcken, R., Wang, G. Z., and Boeckler, F. M. (2012). Kinetic mechanism of p53 oncogenic mutant aggregation and its inhibition.
Zhang, J., Zhang, Z., Brew, K., and Lee, E. Y. (1996). Mutational analysis of the catalytic subunit of muscle protein phosphatase-1. Biochemistry 35, 6276-6282.

1. A method of screening a test compound for modulation of holophosphatase activity comprising:
a) providing a functional and selective holophosphatase comprising a catalytic subunit and at least one regulatory subunit;
b) providing a phosphorylated protein substrate;
c) combining the phosphorylated protein substrate and the holophosphatase and incubating together in the presence or absence of a test compound;
d) measuring dephosphorylation
wherein a variation in dephosphorylation in the presence of the test compound as compared to in the absence of the test compound indicates that the test compound is a modulator of holophosphatase activity.

2. A method according to claim 1 wherein the modulation of holophosphatase activity is inhibition of holophosphatase activity.

3. A method according to claim 1 wherein the modulation of holophosphatase activity is activation of holophosphatase activity.

4. A method according to any preceding claim wherein the holophosphatase activity that is modulated by the test compound is selective holophosphatase activity.

5. A method according to any preceding claim wherein the holophosphatase is purified.

6. A method according to any preceding claim wherein the holophosphatase, the catalytic subunit, the at least one regulatory subunit and/or the phosphorylated substrate is a recombinant protein.

7. A method according to claim 6 wherein the holophosphatase is synthesised by expressing the catalytic subunit and the at least one regulatory subunit in a cell system so as to generate a functional and selective reconstituted form.

8. A method according to any preceding claim wherein the regulatory subunit is a truncated fragment of a naturally occurring regulatory subunit.

9. A method according to any preceding claim wherein the catalytic subunit comprises a Ser/Thr phosphoprotein phosphatase (PPP).

10. A method according to any preceding claim wherein the catalytic subunit comprises a Ser/Thr protein phosphatase 1 subunit (PP1).

11. A method according to any preceding claim wherein the catalytic subunit comprises a PP1c subunit.

12. A method according to any preceding claim wherein the regulatory subunit is selected from R15A and R15B, or fragments thereof.

13. A method according to claim 12 wherein the fragment of a regulatory subunit is selected from R15A³²⁵⁻⁶³⁶ and R15B³⁴⁰⁻⁶⁹⁸.

14. A method according to claim 12 wherein the fragment of a regulatory subunit is selected from R15A^{N} (R15A³²⁵⁻⁵¹²), R15A^{C}(R15A⁵¹³⁻⁶³⁶), R15B^{N}(R15B³⁴⁰⁻⁶³⁵) or R15B^{C} (R15B⁶³⁶⁻⁶⁹⁸).

15. A method according to any of claims 12 to 14 wherein the regulatory subunit or fragment thereof is sufficient for binding inhibitors or activators.

16. A method according to any preceding claim wherein the phosphorylated protein substrate is purified.

17. A method according to any preceding claim wherein the phosphorylated protein substrate is labelled with a detectable label.

18. A method according to any preceding claim wherein the phosphorylation status of the phosphorylated protein substrate may be detected using antibodies which recognise the phosphorylated form of the protein substrate.

19. A method according to any preceding claim wherein the catalytic subunit is provided at a concentration which is sub stoichiometric to the concentration of the phosphorylated protein substrate.

20. A method according to any preceding claim wherein the catalytic subunit is provided at a low concentration.

21. A method according to any preceding claim wherein the catalytic subunit comprises PP1c, and wherein the PP1c is provided at a concentration of less than 1 μM, preferably 0.2 μM, preferably less than 100 nM, preferably 50, 40, 30, 20 or 10 nM.

22. A method according to any preceding claim wherein one or more of the protein components of the screening method may be expressed with an affinity tag.

23. A method according to any preceding claim wherein the catalytic subunit and the at least one regulatory subunit may be endogenous proteins purified from a cell extract.

24. A method according to any preceding claim wherein the method for screening a test compound further comprises performing a confirmatory assay to test for a particular inhibitory/activating activity.

25. A method according to any preceding claim wherein the test compound binds to the regulatory subunit and induces a conformational change in the regulatory subunit.

26. A method according to any preceding claim wherein the test compound is an selective inhibitor.

27. A method for screening for an inhibitor of a holophosphatase comprising providing a test compound and a holophosphatase regulatory subunit under conditions for binding the compound and the regulatory subunit, and detecting a conformational change in the regulatory subunit upon binding of an inhibitor.

28. A method according to claim 27 wherein the conformational change in the regulatory subunit is detected by incubating with a protease, such that if a test compound is a modulator, it alters the sensitivity of the regulatory subunit to protease degradation.

29. A method according to claim 27 wherein the conformational change in the regulatory subunit is detected by a method selected from FRET or NMR.

30. A method of synthesising a reconstituted functional holophosphatase comprising:
a) providing a catalytic subunit at a sub stoichiometric concentration to its substrate;
b) providing a regulatory subunit, or a fragment thereof, which is cognate to the catalytic subunit of a);
c) incubating under conditions to generate a functional holophosphatase.

31. A reconstituted functional holophosphatase produced according to the method of claim 30.

32. A reconstituted functional holophosphatase selected from PP1, PP2, PP3, PP4, PP5, PP6, PP7 having a regulatory subunit comprising binding domains at the carboxy-terminal region sufficient for interaction with the catalytic subunit, binding domains at the amino-terminal region sufficient for recognition of the protein substrate, in combination with an inhibitor binding domain.

33. A reconstituted functional PP1 holophosphatase having a regulatory subunit comprising binding domains at the carboxy-terminal region sufficient for interaction with the catalytic subunit and binding domains at the amino-terminal region sufficient for recognition of the protein substrate.

34. A reconstituted functional PP1 holophosphatase according to claim 33 wherein the regulatory subunit comprises a fragment selected from R15A³²⁵⁻⁶³⁶ or R15B³⁴⁰⁻⁶⁹⁸.

35. A kit for screening test compounds for selective holophosphatase inhibition activity comprising:
a functional holophosphatase in accordance with any of claims 31 to 34, or made according to the method of claim 30; and
a purified labelled phosphorylated holophosphatase protein substrate.

36. A test compound which is an inhibitor of a holophosphatase obtained by a method of screening according to any of claims 1 to 29.

37. A test compound according to claim 36 wherein the test compound is a modulator (inhibitor or activator) of a regulatory subunit.
